(19) United States
(12) Patent Application Publication
(10) Pub. No.: US 2025/0195401 A1
(43) Pub. Date: Jun. 19, 2025

(54) COSMETIC COMPOSITION OF LIPOSOMES CONTAINING AN EXTRACT OF HAEMATOCOCCUS PLUVIALIS, TETRAHEXYLDECYL ASCORBATE AND ECTOIN

(71) Applicant: IMAGE INTERNATIONAL MANUFACTURING, LLC, Lantana, FL (US)

(72) Inventors: Marc A. Ronert, Lantana, FL (US); Margarita M. Cruz Herrera, Lantana, FL (US); Alejandro Llamedo González, Llanera (Asturias) (ES); Rebecca Alonzo Bartolomé, Llanera (Asturias) (ES); Daniel Pando Rodriquez, Llanera (Asturias) (ES)

(21) Appl. No.: 18/985,788

(22) Filed: Dec. 18, 2024

Related U.S. Application Data
(60) Provisional application No. 63/612,195, filed on Dec. 19, 2023.

(51) Int. Cl. A61K 8/9722; A61K 8/14; A61K 8/49; A61K 8/67; A61K 8/99; A61Q 17/04; A61Q 19/08

(52) U.S. Cl. CPC A61K 8/9722; A61K 8/14; A61K 8/49; A61K 8/676; A61K 8/99; A61Q 17/04; A61Q 19/08; A61K 2800/21

(57) ABSTRACT

Embodiments of compositions for cosmetic and personal care use, i.e. applying to skin, particularly for skin care and maintenance, are provided. The cosmetic and personal care use composition(s) preferably includes one or more active ingredients derived, isolated, or extracted from natural resources, such as algae. Embodiments of the cosmetic and personal care use compositions include Haematococcus pluvialis extract, tetrahexyldecyl ascorbate, ectoin, a delivery system, and optionally, one or more additional skin care related functional ingredients. Embodiments of the cosmetic and personal care use compositions include Haematococcus pluvialis extract, tetrahexyldecyl ascorbate, ectoin, an encapsulating delivery vehicle/vesicle, and optionally, one or more additional skin care related functional ingredients.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/612,195, entitled “COSMETIC COMPOSITION OF LIPOSOMES CONTAINING AN EXTRACT OF HAEMATOCOCCUS PLUVIALIS, TETRAHEXYLDECYL ASCORBATE AND ECTOIN”, filed Dec. 19, 2023. The contents of the above referenced application are incorporated herein by reference in its entirety.

### FIELD OF THE INVENTION

The present invention relates to cosmetic and personal care compositions; to cosmetic and personal care compositions comprising natural ingredients; and more particularly, to cosmetic and personal care compositions comprising an extract of Haematococcus pluvialis, tetrahexyldecyl ascorbate, ectoin, and an encapsulating vehicle/vesical which delivers the encapsulated ingredients to a targeted area.

### BACKGROUND OF THE INVENTION

Cosmetic and personal care compositions are well known products worldwide. The global cosmetic industry is valued at over $570 billion, and growing at a yearly rate of close to 4%. In the United States alone, $49 billion is generated by cosmetic sales each year, with the average American spending between $244 and $313 monthly.

Of the many types of cosmetic and personal care compositions, skin care is of vital importance. Consumers understand the need to care for and protect the skin as the first barrier in maintaining health. Deteriorating environmental conditions, such as increased pollution and increasingly aggressive solar radiation, increase the need for adequate skin protection and require cosmetic products to be increasingly stable and effective. On the other hand, consumers demand that at least some of the active ingredients of cosmetic products be products of natural origin. These factors pose a challenge to the cosmetic industry for the development of its products. The present cosmetic and personal care compositions address these needs.

### SUMMARY OF THE INVENTION

Embodiments of compositions for cosmetic and personal care use, i.e. applying to skin, particularly for skin care and maintenance, are provided. The cosmetic and personal care use composition(s) preferably includes one or more active ingredients derived, isolated, or extracted from natural resources, such as algae. Embodiments of the cosmetic and personal care use compositions include Haematococcus pluvialis extract, tetrahexyldecyl ascorbate, ectoin, a delivery system, and optionally, one or more additional skin care related functional ingredients. Embodiments of the cosmetic and personal care use compositions include Haematococcus pluvialis extract, tetrahexyldecyl ascorbate, ectoin, an encapsulating delivery vehicle/vesicle, and optionally, one or more additional skin care related functional ingredients.

Accordingly, it is an objective of the invention to provide cosmetic and personal care compositions.

It is a further objective of the invention to provide embodiments of cosmetic and personal care compositions for use as a sun protectant or sunscreen.

It is yet another objective of the invention to provide cosmetic and personal care compositions comprising at least one active ingredient derived from a natural resource.

It is yet another objective of the invention to provide cosmetic and personal care compositions comprising Haematococcus pluvialis extract, tetrahexyldecyl ascorbate, ectoin.

It is a still further objective of the invention to provide sunscreen composition comprising at least one active ingredient derived from a natural resource.

It is a still further objective of the invention to provide sunscreen composition comprising Haematococcus pluvialis extract, tetrahexyldecyl ascorbate, ectoin.

It is a further objective of the invention to provide an encapsulated cosmetic and personal care composition(s).

It is yet another objective of the invention to provide encapsulated cosmetic and personal care composition(s) comprising Haematococcus pluvialis extract, tetrahexyldecyl ascorbate, ectoin.

It is a still further objective of the invention to provide an aqueous and stable solution of an encapsulating liposome loaded with an extract of Haematococcus pluvialis extract, tetrahexyldecyl ascorbate, ectoin, and one or more additional skin care related functional ingredients.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1A is a cross section of the skin sample; the Stratum corneum, epidermis and dermis can be clearly distinguished;

FIG. 1B is a cross section of the skin sample dyed with DAPI, cell nuclei are stained in blue;

FIG. 2A is a confocal image of the liposomes (red) after penetration in a skin sample (grey);

FIG. 2B is a confocal image of the liposomes (red) after penetration in a skin sample (grey) stained with DAPI (blue);

FIG. 3A is a chart representing the metabolic activity (resazurin reduction) of non-irradiated hOSEC (Healthy, GROUP I), irradiated (UV Control, Group II) or irradiated and treated with either free antioxidant complex (UV free antioxidant complex, Group IV) or encapsulated complex (UV encapsulated antioxidant complex, Group III);

FIG. 3B is the corresponding data of FIG. 3A;

FIG. 4A is a chart representing the tissue damage (LDH leakage) of non-irradiated hOSEC (Healthy), irradiated (UV Control) or irradiated and treated with either free antioxidant complex UV free antioxidant complex) or encapsulated (UV encapsulated antioxidant complex);

FIG. 4B is the corresponding data of FIG. 4A;

FIG. 5A is a chart representing IL-6 secretion of non-irradiated hOSEC (Healthy), irradiated (UV Control) or irradiated and treated with either free antioxidant complex (UV free antioxidant complex) or encapsulated complex (UV encapsulated antioxidant complex);

FIG. 5B is the corresponding data of FIG. 5A;

FIG. 6A is a chart representing IL-8 secretion of non-irradiated hOSEC (Healthy), irradiated (UV Control) or irradiated and treated with either free antioxidant complex (UV free antioxidant complex) or encapsulated complex (UV encapsulated antioxidant complex). Asterisks (*) indicate a statistically significant difference (p<0.05) compared to the Healthy group, hashes (#) compared to the UV Control group, and dollar sign ($) compared to UV free antioxidant complex;

FIG. 6B is the corresponding data of FIG. 6A;

FIG. 7A is a chart representing Matrix Metalloproteinase 9 (MMP-9) secretion of non-irradiated hOSEC (Healthy), irradiated (UV Control) or irradiated and treated with either free antioxidant complex (UV free antioxidant complex) or encapsulated complex (UV encapsulated antioxidant complex). Asterisks (*) indicate a statistically significant difference (p<0.05) compared to the Healthy group, hashes (#) compared to the UV Control group, and dollar sign ($) compared to UV free antioxidant complex;

FIG. 7B is the corresponding data of FIG. 7A;

FIG. 8 is a graphical representation of the results showing ROS levels induction after HEV radiation. *** Represents statistical significance with p value <0.001;

FIG. 9 is a graphical representation of the results showing ROS levels after treatment of RHE with the Example/Composition 10. **** Represents statistical significance with p value <0.0001;

FIG. 10 is a graphical representation of the results showing ROS levels after treatment of RHE with the Example/Composition 14. **** Represents statistical significance with p value <0.0001;

FIG. 11 is a graphical representation of the results showing ROS levels after treatment of RHE with the Example/Composition 12. **** Represents statistical significance with p value <0.0001;

FIG. 12 is a graphical representation of the results showing ROS levels after treatment of RHE with the Example/Composition 13. **** Represents statistical significance with p value <0.0001;

FIG. 13 is a graphical representation of the results showing ROS levels after treatment of RHE with the Example/Composition 11. **** Represents statistical significance with p value <0.0001;

FIG. 14 is a graphical representation of the skin hydration for Example/Composition 10. The Mean and Standard Error of the Mean (SEM) are shown. **** Represents statistical significance with p-value <0,0001;

FIG. 15 is a graphical representation of the skin hydration for Example/Composition 10 after 14 days. The Mean and Standard Error of the Mean (SEM) are shown. **** Represents statistical significance with p-value <0,0001;

FIG. 16 is a graphical representation of the skin erythema for Example/Composition 10. The Mean and Standard Error of the Mean (SEM) are shown. **** Represents statistical significance with p-value <0,0001;

FIG. 17 is a graphical representation of the skin hydration for Example/Composition 14. The Mean and Standard Error of the Mean (SEM) are shown. **** Represents statistical significance with p-value <0,0001;

FIG. 18 is a graphical representation of the skin hydration after 14 days for Example/Composition 14. The Mean and Standard Error of the Mean (SEM) are shown. **** Represents statistical significance with p-value <0,0001;

FIG. 19 is a graphical representation of the skin hydration for Example/Composition 11. The Mean and Standard Error of the Mean (SEM) are shown. **** Represents statistical significance with p-value <0,0001;

FIG. 20 is a graphical representation of the skin gloss for Example/Composition 11. The Mean and Standard Error of the Mean (SEM) are shown. **** Represents statistical significance with p-value <0,0001;

FIG. 21 is a graphical representation of the skin hydration for Example/Composition 12. The Mean and Standard Error of the Mean (SEM) are shown. **** Represents statistical significance with p-value <0,0001;

FIG. 22 is a graphical representation of the skin hydration after 14 days for Example/Composition 12. The Mean and Standard Error of the Mean (SEM) are shown. **** Represents statistical significance with p-value <0,0001;

FIG. 23 is a graphical representation of the skin hydration for Example/Composition 13. The Mean and Standard Error of the Mean (SEM) are shown. **** Represents statistical significance with p-value <0,0001; and

FIG. 24 is a graphical representation of the skin hydration after 14 days for Example/Composition 13. The Mean and Standard Error of the Mean (SEM) are shown. **** Represents statistical significance with p-value <0,0001;

FIG. 25 is a graphical representation of skin tine evenness for Example/Composition 13. The Mean and Standard Error of the Mean (SEM) are shown. **** Represents statistical significance with p-value <0,0001;

FIG. 26A is a graphical representation of the results showing MMP1 induced gene expression after IR radiation. Data are presented as mean+/−standard error of the median (SEM). Statistical significance is depicted as *** statistical p-value <0.001;

FIG. 26B is a graphical representation of the results showing MMP3 induced gene expression after IR radiation. Data are presented as mean+/−standard error of the median (SEM). Statistical significance is depicted as * statistical p-value <0.05;

FIG. 26C is a graphical representation of the results showing MMP9 induced gene expression after IR radiation. Data are presented as mean+/−standard error of the median (SEM);

FIG. 26D is a graphical representation of the results showing MMP1 gene expression levels after treatment of the RHE with Example/Composition 10. Data are presented as mean+/−standard error of the median (SEM). Statistical significance is depicted as ** statistical p-value <0.01;

FIG. 26E is a graphical representation of the results showing MMP3 gene expression levels after treatment of the RHE with Example/Composition 10.

FIG. 26F is a graphical representation of the results showing MMP1 gene expression levels after treatment of the RHE with Example/Composition 10. Data are presented as mean+/−standard error of the median (SEM). Statistical significance is depicted as * statistical p-value <0.05;

FIG. 27A is a graphical representation of the results showing MMP1 induced gene expression after IR radiation. Data are presented as mean+/−standard error of the median (SEM). Statistical significance is depicted as *** statistical p-value <0.001;

FIG. 27B is a graphical representation of the results showing MMP3 induced gene expression after IR radiation. Data are presented as mean+/−standard error of the median (SEM). Statistical significance is depicted as * statistical p-value <0.05;

FIG. 27C is a graphical representation of the results showing MMP9 induced gene expression after IR radiation. Data are presented as mean+/−standard error of the median (SEM);

FIG. 27D is a graphical representation of the results showing MMP1 gene expression levels after treatment of the RHE with Example/Composition 11. Data are presented as mean+/−standard error of the median (SEM). Statistical significance is depicted as *** statistical p-value <0.001;

FIG. 27E is a graphical representation of the results showing MMP3 gene expression levels after treatment of the RHE with Example/Composition 11. Data are presented as mean+/−standard error of the median (SEM). Statistical significance is depicted as ** statistical p-value <0.01;

FIG. 27F is a graphical representation of the results showing MMP1 gene expression levels after treatment of the RHE with Example/Composition 11. Data are presented as mean+/−standard error of the median (SEM). Statistical significance is depicted as * statistical p-value <0.05;

FIG. 28A is a graphical representation of the results showing normalized cell viability after treatment of human keratinocytes with Example/Composition 10 at the indicated dose range, compared to the non-treated control. Data are presented as mean±standard error of the median (SEM). Statistical significance is depicted as * for p<0.05, ** for p<0.01, *** for p<0.001, **** for p<0.0001;

FIG. 28B is a graphical representation of the results showing normalized cell viability after treatment of human keratinocytes with Example/Composition 10 at the indicated dose range, compared to the non-treated control. Data are presented as mean±standard error of the median (SEM). Statistical significance is depicted as * for p<0.05, ** for p<0.01, *** for p<0.001, **** for p<0.0001;

FIG. 28C is a graphical representation of the results showing ROS levels induction after UD exposure. **** Represents statistical significance with p value <0.0001;

FIG. 28D is a graphical representation of the results showing ROS levels after treatment of human keratinocytes with composition 10. Statistical significance is depicted as * for p<0.05;

FIG. 29A is a graphical representation of the results showing normalized cell viability after treatment of human keratinocytes with Example/Composition 11 at the indicated dose range, compared to the non-treated control. Data are presented as mean±standard error of the median (SEM). Statistical significance is depicted as * for p<0.05, ** for p<0.01, *** for p<0.001, **** for p<0.0001;

FIG. 29B is a graphical representation of the results showing normalized cell viability after treatment of human keratinocytes with Example/Composition 11 at the indicated dose range, compared to the non-treated control. Data are presented as mean±standard error of the median (SEM). Statistical significance is depicted as * for p<0.05, ** for p<0.01, *** for p<0.001, **** for p<0.0001;

FIG. 29C is a graphical representation of the results showing ROS levels induction after UD exposure. **** Represents statistical significance with p value <0.0001; and

FIG. 29D is a graphical representation of the results showing ROS levels after treatment of human keratinocytes with Example/Composition 11. Statistical significance is depicted as * for p<0.05 and ** for p<0.01.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

Tables 1-15 provide illustrative embodiments of cosmetic and personal care compositions. The cosmetic and personal care compositions comprise at least, 1) a natural antioxidant (which may exert a photoprotective effect by absorbing the UV energy, reducing the formation of free radicals generated by UV-induced oxidation reaction), preferably an extract of Haematococcus pluvialis, 2) tetrahexyldecyl ascorbate, 3) ectoin, and 4) an encapsulating delivery vehicle/vesicle which delivers the ingredients to a target area, such as the epidermis of a user, and 5) optionally, one or more additional skin care related functional ingredients. The natural antioxidant/extract of Haematococcus pluvialis, tetrahexyldecyl ascorbate, and ectoin may also be referred to as active ingredients.

Haematococcus pluvialis extract: Haematococcus pluvialis is a freshwater species of Chlorophyta (green algae) from the family Haematococcaceae. Haematococcus pluvialis synthesizes and accumulates high levels of astaxanthin, a pink-colored ketocarotenoid with chemical nature of 3,3′ dihydroxy 4,4 diketo-β-carotene (3,3′-dihydroxy-b,b-carotene-4,4′-dione). Astaxanthin is a natural antioxidant. Haematococcus pluvialis may contain the following beneficial properties for the skin including:
Antioxidant capacity that helps protect the skin against free radicals and oxidative stress. This helps prevent cell damage and reduces signs of aging, such as fine lines and wrinkles;
Moisturizing capacity that can help moisturize the skin, which improves its elasticity and softness. It also helps reduce the appearance of dry, flaky skin;
Anti-inflammatory ability that can reduce inflammation in the skin and soothe irritated or sensitive skin;
Luminosity enhancing ability that can help improve the skin's radiance, making it look brighter and more radiant; and
UV protection ability, which can help prevent cell damage and premature aging of the skin.

In certain embodiments, the H. pluvalis extract may include Caprylic Capric Triglyceride and Haematococcus Pluvialis extract. In certain embodiments, the H. pluvalis extract may include Caprylic Capric Triglyceride (greater than 50%, % mass fraction) and Haematococcus Pluvialis Extract (5.0%-9.9%, % mass fraction).

The H. pluvalis extract may be obtained by combining Caprylic/Capric Triglyceride with the Haematococcus Pluvialis microalgae extract following a maceration process in a closed container at cold temperature under specific agitation during a controlled time. The final product is then packed under nitrogen. The microalgae inoculum of Haematococcus Pluvialis is then cultivated in a closed photobioreactor. The bioreactors are provided with appropriate sunlight and CO2. The inoculum multiplies in a specific culture medium and the microalgae biomass is obtained following a certain time of cultivation. The biomass is then harvested, dried and controlled. The biomass is then extracted with ethanol (vegetable based) to obtain the Haematococcus Pluvialis Extract.

Tetrahexyldecyl ascorbate: Tetrahexyldecyl ascorbate is a fat-soluble derivative of vitamin C, having the following properties:
Antioxidant capacity so it helps protect the skin from free radicals that can cause cell damage and accelerate the aging process of the skin;
Depigmenting ability, helping to reduce the appearance of dark spots on the skin, which can improve skin tone and radiance;
Ability to stimulate collagen production so it helps improve skin firmness and elasticity, as well as reduce the appearance of wrinkles and fine lines;
Anti-inflammatory ability that can help reduce inflammation in the skin and soothe irritated or sensitive skin; and

UV protection ability so it can help prevent cell damage and premature aging of the skin.
Ectoin: Ectoin, also Ectoine, is a low molecular, cyclic amino acid derivative produced by many different extremophilic microorganisms.
Chemical name: Ectoine; (4S)-2-methyl-1,4,5,6-tetrahydropyrimidine-4-carboxylic acid
CAS No: 96702-03-3
Molecular formula: C₆H₁₀N₂O₂
Structure:

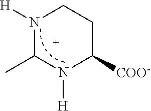

Ectoine belongs to the class of compatible solutes, also called extremolytes (osmolytes from extremophiles; protective small molecules from extremophilic microorganisms). Ectoine is a colorless, crystalline, slightly hygroscopic solid. It is very stable in a wide pH range (1-9) and at high temperatures (6 h at 190° C.).

Ectoin may have the following beneficial cosmetic properties:
Ability to protect against UV rays by acting as a natural filter and also helps prevent inflammation and redness of the skin due to sun exposure;
Increase skin's moisturizing ability by improving the skin's ability to retain moisture and help prevent dry, flaky skin and keep skin soft and smooth;
Anti-inflammatory capacity to help relieve the symptoms of inflammatory skin processes, such as acne and atopic dermatitis;
Protect against free radicals, protect the skin from the actions that cause cell damage and premature aging.

Encapsulating delivery vehicle/vesicle: Hold composition and deliver composition to a desired area of a user. In an illustrative embodiment, the encapsulating delivery vehicle/vesicle is liposomes. The liposome encapsulating delivery vehicle should preferably 1) protect the composition from degradation, 2) maximize delivery of the active ingredient to the deep skin levels, 3) increase the bioavailability of the active ingredient, and 4) provide for greater composition efficiency and stability.

Liposomes: Spherical vesicles with a membrane composed of a double layer of phospholipids, consisting of water-soluble and fat-soluble parts. Preferably, the liposomes are nanocarriers of the composition having a diameter particle size of 50-500 nm. Liposomes are osmotically active and stable and have numerous advantages as a carrier/delivery system, such as their good solubilization power or their ability to increase the stability of a molecule contained in them due to the electrical charge of their surface. In addition, they are biodegradable, biocompatible and non-immunogenic, and exhibit good colloidal, chemical and biological stability.

Embodiments of the liposomes may have a biomimetic membrane with structural components, natural stabilizers, and biomolecule, all derived from algae to provide exosome-like characteristics (defining XOSM technology), and include one or more of the following property characteristics:
ectoin, Haematococcus pluvialis extract, and tetrahexyldecyl ascorbate encapsulated in the liposomes;
algae biomimetic liposomal membrane—fatty acids with long-chain omega 3, omega 6 such as eicosapentaenoic acid (EPA) and eicosatetraenoic acid are present as structural components of the membrane;
phytostherols (from algae) on the membrane act as membrane stabilizers;
nanovesicle with high moisturizing and restorative action;
carotenoids, vitamins and xanthophylls from algae gives natural biomolecules inside the liposome;
algae biomimetic membrane and biomolecules from algae in a small vesicle generates an Exosome-like vesicle.

The liposomes are designed to pass through the Stratum Corneum, reaching the epidermis. Thus, the liposomes provide targeted delivery to the epidermis, passing through the Stratum Corneum. Once at the location, the liposomes provide specific release of the active ingredient/composition into the epidermal layer of the skin.

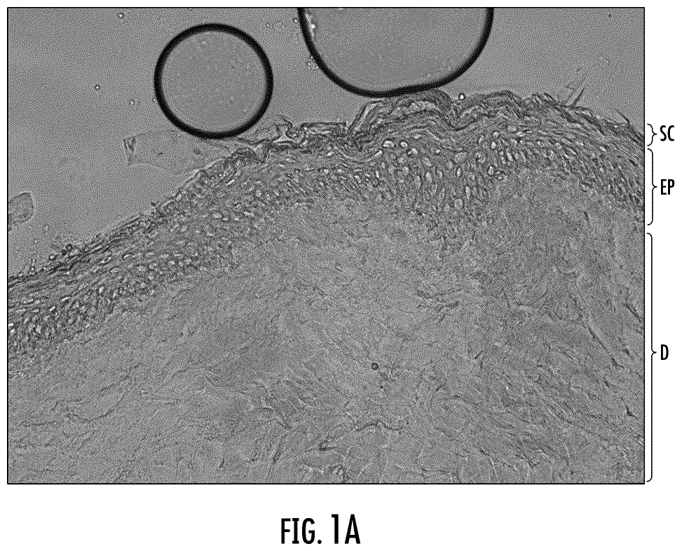

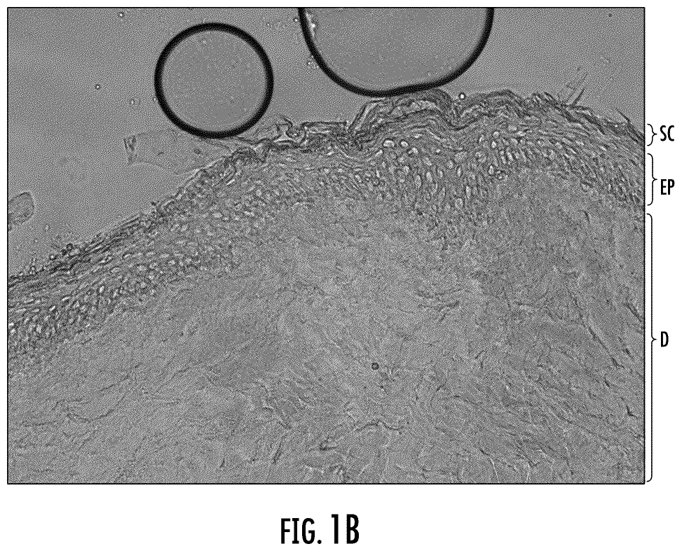

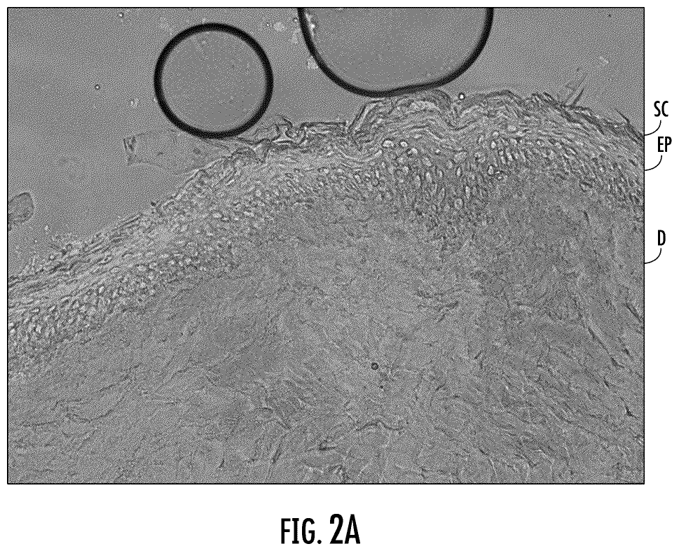

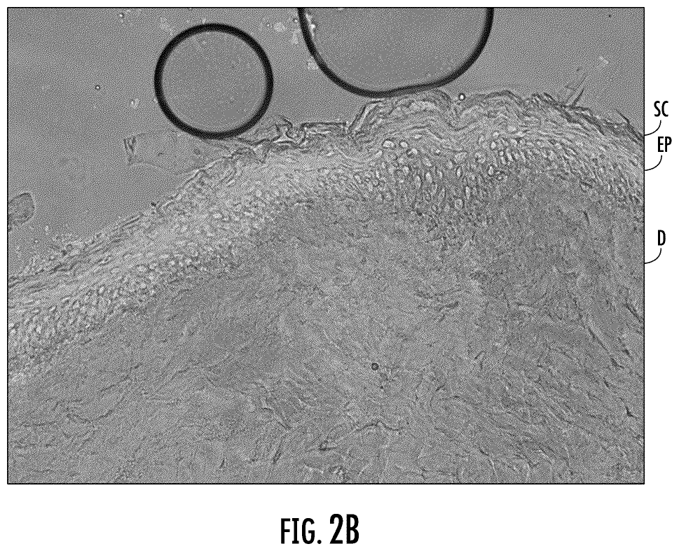

Referring to FIGS. 1A-2B, skin penetration capabilities using examples of liposomes (INDERMAL) are shown. The figures illustrate how the liposomes passed through the Stratum Corneum reaching epidermis, thus providing a targeted delivery to the epidermis passing through the Stratum Corneum. Accordingly, specific release of all active ingredients of the encapsulating liposome loaded with an extract of Haematococcus pluvialis, tetrahexyldecyl ascorbate and ectoin can be specifically released into the epidermal layer of the skin.

To test the skin penetration of the liposomes provided by INDERMAL, the following was undertaken:

Fluorescent liposomes were synthesized including rhodamine-labelled phospholipids (18:1 PE CF) in the membrane of the liposomes. Product was characterized prior to the analysis, to assure that it was in accordance with the specifications regarding size and polydispersity index. Fluorescent liposomes comprised at least an extract of Haematococcus pluvialis, tetrahexyldecyl ascorbate, and ectoin.

Frozen skin samples were hydrated and set to room temperature using a saline buffer. Once the skin was at working conditions, it was mounted on the Franz diffusion cells, and the water bath was set to 37° C. to simulate physiological conditions. 100 μL of encapsulated antioxidant complex were added to the receptor compartment of the Franz diffusion cells, and the compound was left to diffuse for 16 hours. After that time, the skin sample was rinsed off with ultrapure water and fixed with paraformaldehyde (PFA) for 5 hours. Finally, the sample was embedded in OCT, cryopreserved and sectioned using a cryostat, obtaining a cross section of the skin, se FIG. 1A.

These skin sections were dyed with DAPI, a fluorescent stain with specificity for cellular nuclei with an emission maximum at 461 nm (blue) and observed with a confocal microscope. This dye allows for localization of cells throughout the tissue, as it can be observed, see FIG. 1B. The sample was also observed at the emission wavelength of rhodamine, and contrasted with the transmission image of the sample, gaining information regarding the penetration capabilities of the liposomes, see FIG. 2A and FIG. 2B. These images illustrate that the liposomes passed through the Stratum Corneum reaching the epidermis. This liposomal distribution is in accordance with what was expected for the product, which is a targeted delivery to the epidermis passing through the Stratum Corneum. Thereafter, it can be concluded that the liposomes provide a specific release of the active ingredient into the epidermal layer of the skin.

Skin care related functional compounds (may also be referred to as non-active ingredients): Compounds that function as a/an: solvent; emollient; texture enhancing ingredient; UV filter; humectant; skin hydration enhancer; carrier; filler; emulsion stabilizer; opacifying agent; moisturizing agent; emulsifier; antioxidant; stabilizer; conditioning agent, water-binder; viscosity agent; Skin conditioner; preservative; a dye, such as a mineral dye; mild cleansing agent; Skin-softening agent; occlusive; hydrating agent; Fragrance; viscosity-decreasing agent, masking agent; whitening agent; binding agent; buffering agent; pH controller; anti-inflammatory; free radical protectant; luminosity enhancer; collagen stimulator, depigmenting agent; abrasive, anti-caking agent, anti-bulking agent and as an absorbent; pH adjuster; color additive; Skin Protector from UV damage; absorb UV rays; conditioning agent; water-binding properties; short-wave UVB ray absorber; sunscreen agent that works primarily in the UVB range; texture enhancing ingredient; moisturizer; surfactant; synthetic texture-enhancing ingredient; suspending agent; film-former; texture-enhancing thickener; suspending/dispersing agent; viscosity increasing agent; chelating agent; protective and natural skin conditioning; synthetic skin-softening agent; abrasive; absorbent; bulking, viscosity controller; free radical protectant; luminosity enhancer; collagen stimulator; cell generation restorer, elasticity improver. The cosmetic composition may include one or more of each type/category of ingredients and/or any combination of the ingredients, including one or more types of each individual ingredient type or category.

Referring to Table 1, an aqueous and stable solution of liposomes loaded with an extract of Haematococcus pluvialis, tetrahexyldecyl ascorbate and ectoin is provided.

TABLE 1
Cosmetic Composition 1

| Compound | Concentration | |
|---|---|---|
| Haematococcus pluvialis | As required | Antioxidant, |
| extract | | anti-inflammatory, |
| | | moisturizer, |
| | | luminosity |
| | | enhancer, |
| | | UV protectant |
| tetrahexyldecyl ascorbate | As required | Antioxidant, |
| | | anti-inflammatory, |
| | | UV protectant, |
| | | collagen |
| | | stimulator, |
| | | depigmenting |
| Ectoin | As required | Anti-inflammatory, |
| | | UV protectant, |
| | | moisturizer, free |
| | | radical protectant |

Referring to Table 2, an aqueous and stable solution of an encapsulating delivery vehicle/vesicle loaded with an extract of Haematococcus pluvialis, tetrahexyldecyl ascorbate and Ectoin is provided.

TABLE 2
Cosmetic Composition 2

| Compound | Concentration | |
|---|---|---|
| Haematococcus pluvialis | As required | Antioxidant, |
| extract | | anti-inflammatory, |
| | | moisturizer, |
| | | luminosity enhancer, |
| | | UV protectant |
| Tetrahexyldecyl ascorbate | As required | Antioxidant, |
| | | anti-inflammatory, |
| | | UV protectant, |
| | | collagen stimulator, |
| | | depigmenting |
| Ectoin | As required | Anti-inflammatory, |
| | | UV protectant, |
| | | moisturizer, free |
| | | radical protectant |
| Encapsulating delivery | As required | Delivery/penetration |
| vehicle/vesicle | | |

Referring to Table 3, an aqueous and stable solution of an encapsulating liposome loaded with an extract of Haematococcus pluvialis, tetrahexyldecyl ascorbate and ectoin is provided.

TABLE 3
Cosmetic Composition 3

| Compound | Concentration | |
|---|---|---|
| Haematococcus pluvialis | As required | Antioxidant, |
| extract | | anti-inflammatory, |
| | | moisturizer, |
| | | luminosity enhancer, |
| | | UV protectant |
| Tetrahexyldecyl | As required | Antioxidant, |
| ascorbate | | anti-inflammatory, |
| | | UV protectant, |
| | | collagen stimulator, |
| | | depigmenting |
| Ectoin | As required | Anti-inflammatory, |
| | | UV protectant, |
| | | moisturizer, free |
| | | radical protectant |
| Liposome | As required | Delivery/penetration |

Referring to Table 4, an aqueous and stable solution of an encapsulating liposome loaded with an extract of Haematococcus pluvialis, tetrahexyldecyl ascorbate, Ectoin, and one or more additional compounds is provided.

TABLE 4
Cosmetic Composition 4

| Compound | Concentration | |
|---|---|---|
| Haematococcus pluvialis | As required | Antioxidant, |
| extract | | anti-inflammatory, |
| | | moisturizer, |
| | | luminosity enhancer, |
| | | UV protectant |
| Tetrahexyldecyl | As required | Antioxidant, |
| ascorbate | | anti-inflammatory, |
| | | UV protectant, |
| | | collagen stimulator, |
| | | depigmenting |
| Ectoin | As required | Anti-inflammatory, |
| | | UV protectant, |
| | | moisturizer, free |
| | | radical protectant |
| Liposome | As required | Delivery/penetration |
| One or more additional | As required | One or more skin care |
| compounds | | related functional |
| | | compounds. |

Referring to Table 5, an aqueous and stable solution of an encapsulating liposome loaded with an extract of Haematococcus pluvialis, tetrahexyldecyl ascorbate, Ectoin, and one or more skin care related functional compounds, such as: moisturizer, restore cell generation, improve elasticity, UV protectant, emollient, emulsifier, cleansing agent; humectant, is provided.

TABLE 5
Cosmetic Composition 5

| Compound | Concentration | |
|---|---|---|
| Haematococcus pluvialis | As required | Antioxidant, |
| extract | | anti-inflammatory, |
| | | moisturizer, |
| | | luminosity enhancer, |
| | | UV protectant |
| Tetrahexyldecyl ascorbate | As required | Antioxidant, |
| | | anti-inflammatory, |
| | | UV protectant, |
| | | collagen stimulator, |
| | | depigmenting |
| Ectoin | As required | Anti-inflammatory, |
| | | UV protectant, |
| | | moisturizer, free |
| | | radical protectant |
| Liposome | As required | Delivery/penetration |
| Panthenol | As required | Moisturizer, restore |
| | | cell generation, |
| | | improve elasticity, |
| | | UV protectant |
| Phosphatidylcholine | As required | Emollient, |
| | | Emulsifier, |
| | | Cleansing Agent |
| Mannitol | As required | Humectant |
| Glycerine | As required | Humectant, Texture |
| | | Enhancer |
| Metilpropanediol, | As required | Humectant |
| Caprylic/capric | As required | Emollient, Texture |
| triglyceride | | Enhancer |
| Caprylyl glycol | As required | Humectant |
| Stearic acid | As required | Moisturizing agent |
| Palmitic acid | As required | Moisturizer |

Referring to Table 6, an aqueous and stable solution of an encapsulating liposome loaded with an extract of Haematococcus pluvialis, tetrahexyldecyl ascorbate, Ectoin, and one or more skin care related functional compounds, is provided.

TABLE 6
Cosmetic Composition 6

| Compound | Concentration | |
|---|---|---|
| Haematococcus pluvialis | As required | Antioxidant, |
| extract | | anti-inflammatory, |
| | | moisturizer, |
| | | luminosity enhancer, |
| | | UV protectant |
| Tetrahexyldecyl ascorbate | As required | Antioxidant, |
| | | anti-inflammatory, |
| | | UV protectant, |
| | | collagen stimulator, |
| | | depigmenting |
| Ectoin | As required | Anti-inflammatory, |
| | | UV protectant, |
| | | moisturizer, free |
| | | radical protectant |
| Liposome | As required | small vesical, |
| | | spherical in shape, |
| | | having at least one |
| | | lipid layer. Due to |
| | | their hydrophobicity |
| | | and/or hydrophilicity, |
| | | biocompatibility, |
| | | particle size and |
| | | many other properties, |
| | | liposomes can be |
| | | used as a delivery |
| | | system; penetration |
| Panthenol | As required | Moisturizer, restore |
| | | cell generation, |
| | | improve elasticity, |
| | | UV protectant |
| Phosphatidylcholine | As required | Emollient, |
| | | Emulsifier, |
| | | Cleansing Agent |
| Mannitol | As required | Humectant |
| Glycerine | As required | Humectant, Texture |
| | | Enhancer |
| Metilpropanediol | As required | Humectant |
| Caprylic/capric | As required | Emollient, Texture |
| triglyceride | | Enhancer |
| Caprylyl glycol | As required | Humectant |
| Stearic acid | As required | Moisturizing agent |
| Palmitic acid | As required | Moisturizer |
| Phenylpropanol | As required | Preservative |

Referring to Table 7, an aqueous and stable solution of an encapsulating liposome loaded with an extract of Haematococcus pluvialis, tetrahexyldecyl ascorbate, Ectoin, and one or more skin care related functional compounds, is provided.

TABLE 7
Cosmetic Composition 7

| | Concentration | |
|---|---|---|
| | (% by weight with | |
| | respect to the total | |
| | weight of the | |
| Compound | composition) | |
| Haematococcus | between 0.10 and 0.20 | Antioxidant, |
| pluvialis extract | % by weight | anti-inflammatory, |
| | | moisturizer, |
| | | luminosity enhancer, |
| | | UV protectant |
| Tetrahexyldecyl | between 0.05 and 0.15 | Antioxidant, |
| ascorbate | % by weight | anti-inflammatory, |
| | | UV protectant, |
| | | collagen stimulator, |
| | | depigmenting |
| Ectoin | between 0.50 and 1.50 | Anti-inflammatory, |
| | % by weight | UV protectant, |
| | | moisturizer, free |
| | | radical protectant |
| Liposome | As needed | Delivery/penetration |
| Panthenol | between 0.05 and 0.15 | Moisturizer, restore |
| | % by weight | cell generation, |
| | | improve elasticity, |
| | | UV protectant |
| Phosphatidylcholine | between 6.00 and 8.00 | Emollient, Emulsifier, |
| | % by weight | Cleansing Agent |
| Mannitol | between 4.00 and 6.00 | Humectant |
| | % by weight | |
| Glycerine | between 2.60 and 3.80 | Humectant, Texture |
| | % by weight | Enhancer |
| Methylpropanediol, | between 2.00 and 2.80 | Humectant |
| | % by weight | |
| Caprylic/capric | between 1.65% and | Emollient, Texture |
| triglyceride | 2.00% by weight | Enhancer |
| Caprylyl glycol | between 0.30 and 0.50 | Humectant |
| | % by weight | |
| Stearic acid | between 0.25 and 0.45 | Moisturizing agent |
| | % by weight | |
| Palmitic acid | 0.25 and 0.45% by | Moisturizer |
| | weight | |
| Phenylpropanol | 0.06 and 0.10% | Preservative |
| | by weight | |
| Water | to 100.00% by weight | Solvent |

Referring to Table 8, an aqueous and stable solution of an encapsulating liposome loaded with an extract of Haematococcus pluvialis, tetrahexyldecyl ascorbate, ectoin, and one or more skin care related functional compounds, is provided.

TABLE 8
Cosmetic Composition 8

| | Concentration | |
|---|---|---|
| | (% by weight with | |
| | respect to the total | |
| | weight of the | |
| Compound | composition) | |
| Haematococcus | between 0.12 and 0.15% | Antioxidant, |
| pluvialis extract | by weight | anti-inflammatory, |
| | | moisturizer, |
| | | luminosity enhancer, |
| | | UV protectant |
| Tetrahexyldecyl | between 0.10 and 0.12% | Antioxidant, |
| ascorbate | by weight | anti-inflammatory, |
| | | UV protectant, |
| | | collagen stimulator, |
| | | depigmenting |
| Ectoin | between 0.80 and 1.00% | Anti-inflammatory, |
| | by weight | UV protectant, |
| | | moisturizer, free |
| | | radical protectant |
| Liposome | As needed | Delivery/penetration |
| Panthenol | between 0.08 and 0.10% | Moisturizer, restore |
| | by weight | cell generation, |
| | | improve elasticity, |
| | | UV protectant |
| Phosphatidylcholine | between 4.00 and 6.00% | Emollient, |
| | by weight | Emulsifier, |
| | | Cleansing Agent |
| Mannitol | between 2.00 and 4.00% | Humectant |
| | by weight | |
| Glycerine | between 3.00 and 3.50% | Humectant, Texture |
| | by weight | Enhancer |
| Methylpropanediol, | between 2.30 and 2.60% | Humectant |
| | by weight | |
| Caprylic/capric | between 1.70 and 1.90% | Emollient, Texture |
| triglyceride | by weight | Enhancer |
| Caprylyl glycol | between 0.35 and 0.45% | Humectant |
| | by weight | |
| Stearic acid | between 0.30 and 0.40% | Moisturizing agent |
| | by weight | |
| Palmitic acid | between 0.30 and 0.40% | Moisturizer |
| | by weight | |
| Phenylpropanol | between 0.08 and 0.09 ola | Preservative |
| | by weight | |
| Water | to 100.00% by weight | Solvent |

Referring to Table 9, an aqueous and stable solution of an encapsulating liposome loaded with an extract of Haematococcus pluvialis, tetrahexyldecyl ascorbate, ectoin, and one or more skin care related functional compounds, is provided.

TABLE 9
Cosmetic Composition 9

| | Concentration | |
|---|---|---|
| | (% by weight with | |
| | respect to the total | |
| | weight of the | |
| Compound | composition) | |
| Haematococcus | 0.20% by weight | Antioxidant, |
| pluvialis extract | | anti-inflammatory, |
| | | moisturizer, |
| | | luminosity enhancer, |
| | | UV protectant |
| Tetrahexyldecyl | 0.15% by weight | Antioxidant, |
| ascorbate | | anti-inflammatory, |
| | | UV protectant, |
| | | collagen stimulator, |
| | | depigmenting |
| Ectoin | 1.50% by weight | Anti-inflammatory, |
| | | UV protectant, |
| | | moisturizer, free |
| | | radical protectant |
| Liposome | As needed | Delivery/penetration |
| Panthenol | 0.15% by weight | Moisturizer, restore |
| | | cell generation, |
| | | improve elasticity, |
| | | UV protectant |
| Phosphatidylcholine | 8.00% by weight | Emollient, |
| | | Emulsifier, |
| | | Cleansing Agent |
| Mannitol | 6.00% by weight | Humectant |
| Glycerine | 3.80% by weight | Humectant, Texture |
| | | Enhancer |
| Methylpropanediol, | 2.80% by weight | Humectant |
| Caprylic/capric | 2.00% by weight | Emollient, Texture |
| triglyceride | | Enhancer |
| Caprylyl glycol | 0.50% by weight | Humectant |
| Stearic acid | 0.45% by weight | Moisturizing agent |
| Palmitic acid. | 0.45% by weight | Moisturizer |
| Phenylpropanol | 0.10% by weight | Preservative |
| Water | to 100.00% by weight | Solvent |
| Liposome | As needed | Delivery/penetration |

Referring to Table 10, an aqueous and stable solution of an encapsulating liposome loaded with an extract of Haematococcus pluvialis, tetrahexyldecyl ascorbate, ectoin, and one or more skin care related functional compounds, such as: solvent, texture enhancer, emollient, moisturizing ingredient, emulsifier, surfactant, silicone, occlusive/opacifying agent, thickener, stabilizer, texturizing agent, film former, preservative, mineral dye, sunscreen booster, antioxidant, chelating agent, absorbent, abrasive, a thickener, preservative, humectant, skin conditioner, cleansing agent, fragrance, restore cell generation, improve elasticity, UV protectant, pH adjuster/stabilizer, chelating agent, mineral, is provided.

TABLE 10
Cosmetic Composition 10

| | Concentration | |
|---|---|---|
| | (% by weight with | |
| | respect to the | |
| | total weight of the | |
| Compound | composition) | |
| Water | 25-50 | Solvent |
| C13-15 Alkane | 5-15.0 | Texture enhancer |
| Zinc Oxide | 10.5 | UV filter |
| Caprylic/Capric | 5-15.0 | Emollient, Texture |
| Triglyceride | | Enhancer |
| Butyloctyl Salicylate | 1.9-4.9 | |
| Squalane | 1.9-4.9 | Moisturizing |
| | | ingredient |
| Titanium Dioxide | 3.7 | |
| Polyglyceryl-3 | 1.9-4.9 | Emulsifier |
| Polyricinoleate | | |
| Coco-Caprylate/Caprate | 1.9-4.9 | Emollient |
| Polyglyceryl-10 | 1.9-4.9 | Emulsifier and |
| Dioleate | | surfactant |
| Cetyl Dimethicone | 0.75-2.5 | Silicone, Emollient, |
| | | Occlusive/Opacifying |
| | | Agent |
| Disteardimonium | 0.75-2.5 | Thickener, |
| Hectorite | | stabilizer |
| Polyhydroxystearic Acid | 0.75-2.5 | Texturizing agent, |
| | | thickener, emollient |
| | | and film former |
| Glycerin | 0.75-2.5 | Preservative |
| Phenoxyethanol | 0.75-2.5 | Preservative |
| Propanediol | 0.75-2.5 | Solvent, Texture |
| | | Enhancer |
| Stearic Acid | 0.75-2.5 | Emollient, |
| | | surfactant, and |
| | | emulsifier |
| Iron Oxide (CI 77492) | 0.75-2.5 | Mineral dye |
| Styrene/Acrylates | 0.75-2.5 | Opacifying Agent, |
| Copolymer | | Sunscreen Booster, |
| | | Film Former |
| Tocopheryl Acetate | 0.1-0.9 | Antioxidant |
| Acetyl Zingerone | 0.1-0.9 | Antioxidant, |
| | | Chelating Agent |
| Hydrated Silica | 0.1-0.9 | Texture Enhancer, |
| | | Absorbent, |
| | | Occlusive/Opacifying |
| | | Agent |
| Alumina | 0.1-0.9 | Abrasive, a |
| | | thickener, and an |
| | | absorbent |
| Ethylhexylglycerin | 0.1-0.9 | Preservative, |
| | | Humectant |
| Iron Oxide (CI 77491) | ≤0.1% | Mineral dye |
| Allantoin | ≤0.1% | Skin conditioner |
| Phosphatidylcholine | ≤0.1% | Emollient, |
| | | Emulsifier, |
| | | Cleansing Agent |
| Mannitol | ≤0.1% | Humectant |
| Iron Oxide (CI 77499) | ≤0.1% | Mineral dye |
| Vanilla Planifolia | ≤0.1% | Antioxidant, |
| Fruit Extract | | Fragrance |
| Enteromorpha Compressa | ≤0.1% | Antioxidant, Texture |
| Extract | | Enhancer |
| Triethoxycaprylylsilane | ≤0.1% | Silicone, Texture |
| | | Enhancer |
| Methyl Propanediol | ≤0.1% | Solvent, Humectant |
| Ectoin | ≤0.1% | Anti-inflammatory, |
| | | UV protectant, |
| | | moisturizer, free |
| | | radical protectant |
| Camellia Sinensis Leaf | ≤0.1% | Antioxidant |
| Extract | | |
| Silybum Marianum Fruit | ≤0.1% | Antioxidant |
| Extract | | |
| Ocimum Sanctum Leaf | ≤0.1% | Antioxidant |
| Extract | | |
| Caprylylglycol | ≤0.1% | Humectant |
| Palmitic Acid | ≤0.1% | Emollient |
| Haematococcus Pluvialis | ≤0.1% | Antioxidant, |
| Extract | | anti-inflammatory, |
| | | moisturizer, |
| | | luminosity enhancer, |
| | | UV protectant |
| Sodium Benzoate | ≤0.1% | Preservative |
| Potassium Sorbate | ≤0.1% | Preservative |
| Tetrahexyldecyl | ≤0.1% | Antioxidant, |
| Ascorbate | | anti-inflammatory, |
| | | UV protectant, |
| | | collagen stimulator, |
| | | depigmenting |
| Panthenol | ≤0.1% | Humectant, |
| | | moisturizer, |
| | | restore cell |
| | | generation, improve |
| | | elasticity, |
| | | UV protectant |
| Tocopherol | ≤0.1% | Antioxidant |
| Citric Acid | ≤0.1% | pH |
| | | Adjuster/Stabilizer, |
| | | Chelating Agent |
| Phenylpropanol | ≤0.1% | Preservative |
| Magnesium Oxide | ≤0.1% | Mineral |
| Liposome | As needed | Delivery/penetration |

Referring to Table 11, an aqueous and stable solution of an encapsulating liposome loaded with an extract of Haematococcus pluvialis, tetrahexyldecyl ascorbate, Ectoin, and one or more skin care related functional compounds, such as: solvent, UV filter, texturizing agent, thickener, emollient and film former, absorbs ultraviolet (UV) light; absorbs short-wave UVB rays, moisturizing agent, emulsifier, and penetration enhancer, humectant, antioxidant, skin conditioner, stabilizer, dispersing agent, treatment agent of filler, texture Enhancer, film-forming agent, abrasive, absorbent, chelating agent, occlusive/opacifying agent, Preservative, gelling agent that thickens, emulsifies, and stabilizes, viscosity agent, emollient, cleansing agent, mineral dye, fragrance, thickening agent, Anti-inflammatory, UV protectant, moisturizer, free radical protectant, pH adjuster/stabilizer, chelating agent, antioxidant, is provided.

TABLE 11
Cosmetic Composition 11

| | Concentration | |
|---|---|---|
| | (% by weight with | |
| | respect to the total | |
| | weight of the | |
| Compound | composition) | |
| Water | 25-50 | Solvent |
| Zinc Oxide | 9.3 | UV filter |
| Cyclopentasiloxane | 5-10.0 | Emollient, |
| | | lubricant, and |
| | | solvent |
| Polyhydroxystearic Acid | 5-10.0 | Texturizing agent, |
| | | thickener, emollient |
| | | and film former |
| Ethylhexyl Salicylate | 5 | UV Filter |
| Homosalate | 5 | Absorbs ultraviolet |
| | | (UV) light; absorbs |
| | | short-wave UVB rays |
| Dimethicone | 1.9-4.9 | Emollient |
| Coconut Alkanes | 1.9-4.9 | Emollient |
| Stearic Acid | 1.9-4.9 | Moisturizing agent |
| Lecithin | 1.9-4.9 | Emollient, |
| | | emulsifier, and |
| | | penetration enhancer |
| Aloe Barbadensis Leaf | 1.9-4.9 | Humectant, |
| Juice | | Antioxidant |
| Isohexadecane | 0.75-2.5 | Emollient, skin |
| | | conditioner |
| Isododecane | 0.75-2.5 | Emollient |
| Magnesium Sulfate, | 0.75-2.5 | Stabilizer |
| Heptahydrate | | |
| Dimethicone/PEG-10/15 | 0.75-2.5 | Stabilizer, |
| Crosspolymer | | dispersing agent, |
| | | emulsifier |
| Polyglyceryl-3 | 0.75-2.5 | Treatment agent of |
| Polydimethylsiloxyethyl | | filler |
| Dimethicone | | |
| Glycerin | 0.75-2.5 | Humectant, Texture |
| | | Enhancer |
| Polysilicone-11 | 0.1-0.9 | Film-forming agent |
| Cetearyl Olivate | 0.1-0.9 | Emulsifier |
| Caprylic/Capric | 0.1-0.9 | Emollient, Texture |
| Triglyceride | | Enhancer |
| Silica | 0.1-0.9 | Abrasive, absorbant |
| Acetyl Zingerone | 0.1-0.9 | Antioxidant, |
| | | Chelating Agent |
| Triethoxysilylethyl | 0.1-0.9 | Occlusive/Opacifying |
| Polydimethylsiloxyethyl | | Agent |
| Hexyl Dimethicone | | |
| Phenoxyethanol | 0.1-0.9 | Preservative |
| Sorbitan Olivate | 0.1-0.9 | Emulsifier and |
| | | surfactant |
| Hydroxyethyl | 0.1-0.9 | Gelling agent that |
| Acrylate/Sodium | | thickens, |
| Acryloyldimethyl | | emulsifies, and |
| Taurate Copolymer | | stabilizes |
| Caprylyl Glycol | 0.1-0.9 | Preservative |
| Squalane | 0.1-0.9 | Moisturizer |
| Coco-Caprylate/Caprate | 0.1-0.9 | Emollient |
| Ethylhexylglycerin | 0.1-0.9 | Preservative, |
| | | Humectant |
| Hexylene Glycol | 0.1-0.9 | Solvent, Texture |
| | | Enhancer, viscosity |
| | | agent |
| Octyldodecanol | 0.1-0.9 | Occlusive/Opacifying |
| | | Agent, Emollient, |
| | | Texture Enhancer |
| Phosphatidylcholine | ≤0.1% | Emollient, |
| | | Emulsifier, |
| | | Cleansing Agent |
| CI 77492 (Iron Oxides) | ≤0.1% | Mineral dye |
| Vanilla Planifolia | ≤0.1% | Antioxidant, |
| Fruit Extract | | Fragrance |
| Polysorbate 60 | ≤0.1% | Thickening agent |
| Mannitol | ≤0.1% | Humectant |
| Dimethicone/Vinyl | ≤0.1% | Texture enhancer |
| Dimethicone | | |
| Crosspolymer | | |
| Camellia Sinensis Leaf | ≤0.1% | Antioxidant |
| Extract | | |
| Methyl Propanediol | ≤0.1% | Solvent, Humectant |
| Dipropylene Glycol | ≤0.1% | Texture Enhancer, |
| | | Solvent |
| CI 77491 (Iron Oxides) | ≤0.1% | Mineral dye |
| Sorbitan Isostearate | ≤0.1% | Cleansing Agent, |
| | | Emulsifier |
| Sodium Hyaluronate | ≤0.1% | Humectant |
| ECTOIN | ≤0.1% | Anti-inflammatory, |
| | | UV protectant, |
| | | moisturizer, free |
| | | radical protectant |
| Sodium Citrate | ≤0.1% | pH |
| | | Adjuster/Stabilizer, |
| | | Chelating Agent, |
| | | Antioxidant, |
| | | Preservative |
| Palmitic Acid | ≤0.1% | Emollient, cleansing |
| | | agent, |
| | | Occlusive/Opacifying |
| | | Agent, and texture |
| | | enhancer |
| Haematococcus Pluvialis | ≤0.1% | Antioxidant, anti- |
| Extract | | inflammatory, |
| | | moisturizer, |
| | | luminosity enhancer, |
| | | UV protectant |
| Tetrahexyldecyl | ≤0.1% | Antioxidant, anti- |
| Ascorbate | | inflammatory, UV |
| | | protectant, collagen |
| | | stimulator, |
| | | depigmenting |
| Panthenol | ≤0.1% | Humectant |
| Phenylpropanol | ≤0.1% | Preservative, |
| | | Solvent |
| Tocopherol | ≤0.1% | Antioxidant |
| Sorbitan Oleate | ≤0.1% | Cleansing Agent, |
| | | Emulsifier |
| Sodium Benzoate | ≤0.1% | Preservative |
| Potassium Sorbate | ≤0.1% | Preservative |
| Liposome | As needed | Delivery/penetration |

Referring to Table 12, an aqueous and stable solution of an encapsulating liposome loaded with an extract of Haematococcus pluvialis, tetrahexyldecyl ascorbate, ectoin, and one or more skin care related functional compounds, such as: emulsion stabilizer, film-former, and texture-enhancing thickener, anti-foaming agent, skin protectant, skin conditioning agent, humectant, texture enhancer, emollient, emulsifier, and penetration enhancer, antioxidant, treatment agent of filler, skin-softening agent and skin conditioner, preservative, surfactant, rheology modifier, moisturizing ingredient, fragrance, antioxidant, solvent, thickening agent, pH-adjuster-stabilizer, restore cell generation, improve elasticity, UV protectant, is provided.

TABLE 12
Cosmetic Composition 12

| | Concentration | |
|---|---|---|
| | (% by weight with | |
| | respect to the total | |
| | weight of the | |
| Compound | composition) | |
| Water | 25-50 | Solvent |
| Cyclopentasiloxane | 15-20 | Emollient, |
| | | lubricant, and |
| | | solvent |
| Zinc Oxide | 15-20 | UV filter |
| Dicaprylyl Carbonate | 5-10.0 | Skin-conditioning |
| | | agent, emollient, |
| | | and solvent |
| Polyhydroxystearic Acid | 5-10.0 | Emulsion stabilizer, |
| | | film-former, and |
| | | texture-enhancing |
| | | thickener |
| Dimethicone | 1.9-4.9 | Anti-foaming agent, |
| | | skin protectant, |
| | | skin conditioning |
| | | agent |
| Glycerin | 1.9-4.9 | Humectant, Texture |
| | | Enhancer |
| Lecithin | 1.9-4.9 | Emollient, |
| | | emulsifier, and |
| | | penetration enhancer |
| Isohexadecane | 1.9-4.9 | Emollient, skin |
| | | conditioner |
| Aloe Barbadensis Leaf | 1.9-4.9 | Humectant, |
| Juice | | Antioxidant |
| Polyglyceryl-3 | 0.75-2.5 | Treatment agent of |
| Polydimethylsiloxyethyl | | filler |
| Dimethicone | | |
| Dimethicone/PEG-10/15 | 0.75-2.5 | Emulsion stabilizer |
| Crosspolymer | | |
| Triethoxysilylethyl | 0.75-2.5 | Skin-softening agent |
| Polydimethylsiloxyethyl | | and skin conditioner |
| Hexyl Dimethicone | | |
| Magnesium Sulfate, | 0.1-0.9 | Preservative |
| heptahydrate | | |
| Acetyl Zingerone | 0.1-0.9 | Antioxidant |
| Phenoxyethanol | 0.1-0.9 | Preservative |
| Caprylic/Capric | 0.1-0.9 | Skin conditioner |
| Triglyceride | | |
| Caprylyl Glycol | 0.1-0.9 | Humectant |
| Ethylhexylglycerin | 0.1-0.9 | Preservative, |
| | | Humectant |
| Hexylene Glycol | 0.1-0.9 | Surfactant |
| Hydroxyethyl | 0.1-0.9 | Rheology Modifier |
| Acrylate/Sodium | | |
| Acryloyldimethyl | | |
| Taurate Copolymer | | |
| Squalane | ≤0.1% | Moisturizing |
| | | ingredient |
| Phosphatidylcholine | ≤0.1% | Skin conditioning |
| | | agent, emulsifier |
| | | and surfactant |
| Mannitol | ≤0.1% | Humectant |
| Vanilla Planifolia | ≤0.1% | Antioxidant, |
| Fruit Extract | | Fragrance |
| Camellia Sinensis Leaf | ≤0.1% | Antioxidant |
| Extract | | |
| Methyl Propanediol | ≤0.1% | Solvent |
| Sodium Hyaluronate | ≤0.1% | Humectant |
| Dipropylene Glycol | ≤0.1% | Humectant |
| Polysorbate 60 | ≤0.1% | Thickening agent |
| ectoin | ≤0.1% | Anti-inflammatory, |
| | | UV protectant, |
| | | moisturizer, free |
| | | radical protectant |
| Sodium Citrate | ≤0.1% | pH-adjuster- |
| | | stabilizer |
| Sorbitan Isostearate | ≤0.1% | Moisturizing and |
| | | conditioning |
| | | ingredient, |
| | | emulsifier |
| Tocopherol | ≤0.1% | Antioxidant |
| Stearic Acid | ≤0.1% | Moisturizing agent |
| Palmitic Acid | ≤0.1% | Moisturizer |
| Haematococcus Pluvialis | ≤0.1% | Antioxidant, |
| Extract | | anti-inflammatory, |
| | | moisturizer, |
| | | luminosity enhancer, |
| | | UV protectant |
| Tetrahexyldecyl | ≤0.1% | Antioxidant, |
| Ascorbate | | anti-inflammatory, |
| | | UV protectant, |
| | | collagen stimulator, |
| | | depigmenting |
| Panthenol | ≤0.1% | Humectant, |
| | | Moisturizer, restore |
| | | cell generation, |
| | | improve elasticity, |
| | | UV protectant |
| Phenylpropanol | ≤0.1% | Preservative |
| Sodium Benzoate | ≤0.1% | Preservative |
| Potassium Sorbate | ≤0.1% | Preservative |
| Liposome | As needed | Delivery/penetration |

Referring to Table 13, an aqueous and stable solution of an encapsulating liposome loaded with an extract of Haematococcus pluvialis, tetrahexyldecyl ascorbate, Ectoin, and one or more skin care related functional compounds, such as: solvent; emollient; texture enhancing ingredient; UV filter; humectant; skin hydration enhancer; carrier; filler; emulsion stabilizer; opacifying agent; moisturizing agent; antioxidant; emulsifier; stabilizer; conditioning agent, water-binder; viscosity agent; Skin conditioner; preservative; Mineral dye; mild cleansing agent; skin-softening agent; occlusive; hydrating agent; fragrance; viscosity-decreasing agent, masking agent; whitening agent; binding agent; buffering agent; pH controller; Anti-inflammatory; free radical protectant; luminosity enhancer; collagen stimulator, depigmenting agent; abrasive, anti-caking agent, anti-bulking agent and as an absorbent; pH adjuster; color additive, is provided.

TABLE 13
Cosmetic Composition 13:

| | Concentration | |
|---|---|---|
| | (% by weight with | |
| | respect to the | |
| | total weight of | |
| Compound | the composition) | |
| Water | 25-50 | Solvent |
| Cyclopentasiloxane | 15-20 | Emollient, Solvent |
| C12-15 Alkyl Benzoate | 5-10.0 | Emollient and |
| | | texture enhancing |
| | | ingredient |
| Zinc Oxide | 6 | UV filter |
| Dimethicone | 5-10.0 | Emollient |
| Titanium Dioxide | 4.7 | UV Filter |
| Glycerin | 1.9-4.9 | Humectant, Texture |
| | | Enhancer |
| Cyclohexasiloxane | 1.9-4.9 | Enhance skin |
| | | hydration, carrier |
| Polyglyceryl-3 | 1.9-4.9 | Treatment agent of |
| Polydimethylsiloxyethyl | | filler |
| Dimethicone | | |
| Dimethicone/PEG-10/15 | 0.75-2.5 | Emulsion stabilizer |
| Crosspolymer | | |
| Aluminum Hydroxide | 0.75-2.5 | Opacifying agent |
| Stearic Acid | 0.75-2.5 | Moisturizing agent |
| Sodium Chloride | 0.75-2.5 | |
| Cetearyl Olivate | 0.1-0.9 | Emulsifier |
| Acetyl Zingerone | 0.1-0.9 | Anitoxidant |
| Polyglyceryl-4 | 0.1-0.9 | Emulsifier and |
| Isostearate | | stabilizer |
| Cetyl PEG/PPG-10/1 | 0.1-0.9 | Stabilizer, |
| Dimethicone | | conditioning agent |
| | | and emulsifier, |
| | | provides emollient, |
| | | water-binding |
| | | properties |
| Hexyl Laurate | 0.1-0.9 | Emollient, solvent, |
| | | and viscosity agent |
| Dimethicone/Polyglycerin- | 0.1-0.9 | Skin conditioner |
| 3 Crosspolymer | | |
| Phenoxyethanol | 0.1-0.9 | P Skin conditioner |
| | | reservative |
| Iron Oxide (CI 77492) | 0.1-0.9 | Mineral dye |
| Sorbitan Olivate | 0.1-0.9 | Emulsifier and mild |
| | | cleansing agent. |
| Caprylic/Capric | 0.1-0.9 | Skin conditioner |
| Triglyceride | | |
| Triethoxysilylethyl | 0.1-0.9 | Skin-softening agent |
| Polydimethylsiloxyethyl | | and occlusive. |
| Hexyl Dimethicone | | |
| Caprylyl Glycol | 0.1-0.9 | Humectant |
| Ethylhexylglycerin | 0.1-0.9 | Preservative |
| Hexylene Glycol | 0.1-0.9 | Solvent |
| Dimethicone/Vinyl | 0.1-0.9 | Skin conditioner |
| Dimethicone Crosspolymer | | |
| Tocopheryl Acetate | ≤0.1% | Antioxidant |
| Iron Oxide (CI 77491) | ≤0.1% | Mineral dye |
| Phosphatidylcholine | ≤0.1% | Emulsifier, |
| | | emollient, and |
| | | hydrating agent |
| Mannitol | ≤0.1% | Humectant |
| Iron Oxide (CI 77499) | ≤0.1% | Mineral dye |
| Vanilla Planifolia Fruit | ≤0.1% | Antioxidant, |
| Extract | | Fragrance |
| Camellia Sinensis Leaf | ≤0.1% | Antioxidant |
| Extract | | |
| Dipropylene Glycol | ≤0.1% | Solvent, viscosity- |
| | | decreasing agent, |
| | | masking agent, and |
| | | fragrance |
| Titanium Dioxide (CI | ≤0.1% | Whitening agent and |
| 77891) | | UV protectant |
| Benzoic Acid | ≤0.1% | Preservative |
| Methyl Propanediol | ≤0.1% | Humectant glycol |
| Triethoxycaprylylsilane | ≤0.1% | Binding agent and |
| | | emulsion stabilizer |
| Sodium Citrate | ≤0.1% | Buffering agent, |
| | | control pH |
| ECTOIN | ≤0.1% | Anti-inflammatory, |
| | | UV protectant, |
| | | moisturizer, free |
| | | radical protectant |
| Stearic Acid | ≤0.1% | Moisturizing agent |
| Palmitic Acid | ≤0.1% | Moisturizing agent |
| Haematococcus Pluvialis | ≤0.1% | Antioxidant, anti- |
| Extract | | inflammatory, |
| | | moisturizer, |
| | | luminosity enhancer, |
| | | UV protectant |
| Tocopherol | ≤0.1% | Antioxidant |
| Tetrahexyldecyl Ascorbate | ≤0.1% | Antioxidant, anti- |
| | | inflammatory, UV |
| | | protectant, collagen |
| | | stimulator, |
| | | depigmenting |
| Panthenol | ≤0.1% | Humectant |
| Phenylpropanol | ≤0.1% | Preservative |
| Alumina | ≤0.1% | Abrasive, anti- |
| | | caking agent, anti- |
| | | bulking agent and as |
| | | an absorbent. |
| Magnesium Oxide | ≤0.1% | pH adjuster |
| Sodium Benzoate | ≤0.1% | Preservative |
| Potassium Sorbate | ≤0.1% | Preservative |
| Pentaerythrityl Tetra-di- | ≤0.1% | Antioxidant |
| t-butyl | | |
| Hydroxyhydrocinnamate | | |
| Sodium Ferrocyanide | ≤0.1% | Color additive |
| Liposome | As needed | Delivery/penetration |

Referring to Table 14, an aqueous and stable solution of an encapsulating liposome loaded with an extract of Haematococcus pluvialis, tetrahexyldecyl ascorbate, ectoin, and one or more skin care related functional compounds, such as: solvent; skin protector from UV damage; absorb UV rays; stabilizer, conditioning agent; emulsifier; water-binding properties; short-wave UVB ray absorber; UV filter; emollient; sunscreen agent that works primarily in the UVB range; texture enhancing ingredient; skin elasticity improver, moisturizer; surfactant; synthetic texture-enhancing ingredient; humectant; antioxidant; preservative; emulsion stabilizer; film-former; texture-enhancing thickener; suspending/dispersing agent; viscosity increasing agent; chelating agent; protective and natural skin conditioning; synthetic skin-softening agent; abrasive; absorbent; anti-caking; bulking, opacifying, viscosity controller; fragrance; anti-inflammatory; free radical protectant; luminosity enhancer; collagen stimulator; depigmenting agent; cell generation restorer, elasticity improver, is provided.

TABLE 14
Cosmetic Composition 14

| | Concentration | |
|---|---|---|
| | (% by weight with | |
| | respect to the total | |
| | weight of the | |
| Compound | composition) | |
| Water | 25-50 | Solvent |
| Octocrylene | 9.9 | Protect skin from UV |
| | | damage; absorb UV |
| | | rays, |
| Cetyl PEG/PPG-10/1 | 5-10.0 | Stabilizer, |
| Dimethicone | | conditioning agent |
| | | and emulsifier, |
| | | provides emollient, |
| | | water-binding |
| | | properties |
| Homosalate | 7.3 | Absorbs ultraviolet |
| | | (UV) light; absorbs |
| | | short-wave UVB rays |
| Zinc Oxide | 7 | UV Filter |
| Hydrogenated | 5-10.0 | Emollient and |
| Ethylhexyl Olivate | | conditioning |
| | | ingredient |
| Ethylhexyl Salicylate | 5 | Sunscreen agent that |
| | | works primarily in |
| | | the UVB range |
| C12-15 Alkyl Benzoate | 5-10.0 | Emollient and texture |
| | | enhancing ingredient |
| Theobroma Cacao Seed | 1.9-4.9 | Improve skin |
| Butter | | elasticity, |
| | | moisturize, and |
| | | protect the skin |
| Hydrogenated Castor | 1.9-4.9 | Surfactant, |
| Oil | | emulsifier, and |
| | | emollient |
| C13-15 Alkane | 1.9-4.9 | Synthetic texture- |
| | | enhancing ingredient |
| | | and solvent |
| Aloe Barbadensis Leaf | 1.9-4.9 | Humectant, |
| Juice | | Antioxidant |
| Ethylhexyl sterate | 1.9-4.9 | Emulsifier, |
| | | Emollient, Texture |
| | | Enhancer |
| Isoamyl Laurate | 1.9-4.9 | Emollient |
| Glycerin | 0.75-2.5 | Emollient |
| Ethylhexyl Palmitate | 0.75-2.5 | Emollient |
| Phenoxyethanol | 0.75-2.5 | Preservative |
| Euphorbia Cerifera | 0.75-2.5 | Suspending agent, |
| Wax | | emulsion stabilizer, |
| | | film-former, and |
| | | texture-enhancing |
| | | thickener |
| Polyhydroxystearic | 0.1-0.9 | Suspending/Dispersing |
| Acid | | Agent |
| Sodium Chloride | 0.1-0.9 | Viscosity increasing |
| | | agent |
| Acetyl Zingerone | 0.1-0.9 | Antioxidant, |
| | | Chelating Agent |
| Caprylic/Capric | 0.1-0.9 | Emollient, |
| Triglyceride | | antioxidant, solvent, |
| | | dispersing agent |
| Hydrogenated Olive | 0.1-0.9 | Surfactant |
| Oil Unsaponifiables | | |
| Tocopheryl Acetate | 0.1-0.9 | Antioxidant, |
| | | protective and |
| | | natural skin |
| | | conditioning |
| Ethylhexylglycerin | 0.1-0.9 | Synthetic skin- |
| | | softening agent |
| Hydrated Silica | 0.1-0.9 | Abrasive, Absorbent, |
| | | Anti-caking, Bulking, |
| | | Opacifying, Viscosity |
| | | Controller |
| Phosphatidylcholine | ≤0.1% | Emollient, |
| | | Emulsifier, Cleansing |
| | | Agent |
| Mannitol | ≤0.1% | Humectant |
| Vanilla Planifolia | ≤0.1% | Antioxidant, |
| Fruit Extract | | Fragrance |
| Methyl Propanediol | ≤0.1% | Humectant |
| Benzoic Acid | ≤0.1% | Preservative |
| ectoin | ≤0.1% | Anti-inflammatory, UV |
| | | protectant, |
| | | moisturizer, free |
| | | radical protectant |
| Palmitic Acid | ≤0.1% | Moisturizer |
| Caprylylglycol | ≤0.1% | Preservative |
| Stearic Acid | ≤0.1% | Moisturizing agent |
| Haematococcus | ≤0.1% | Antioxidant, anti- |
| Pluvialis Extract | | inflammatory, |
| | | moisturizer, |
| | | luminosity enhancer, |
| | | UV protectant |
| Tetrahexyldecyl | ≤0.1% | Antioxidant, anti- |
| Ascorbate | | inflammatory, UV |
| | | protectant, collagen |
| | | stimulator, |
| | | depigmenting |
| Panthenol | ≤0.1% | Humectant, |
| | | Moisturizer, restore |
| | | cell generation, |
| | | improve elasticity, |
| | | UV protectant |
| Phenylpropanol | ≤0.1% | Preservative |
| Pentaerythrityl | ≤0.1% | Antioxidant |
| Tetra-di-t-butyl- | | |
| Hydroxyhydrocinnamate | | |
| Liposome | As needed | Delivery/penetration |

Referring to Table 15, an aqueous and stable solution, in the form of a mist, of an encapsulating liposome loaded with an extract of Haematococcus pluvialis, tetrahexyldecyl ascorbate, ectoin, and one or more skin care related functional compounds, such as a solvent, UV filter, emollient, skin conditioner, film former, antioxidant, chelating agent, humectant, texture enhancer, emulsifier, cleansing agent, anti-inflammatory, UV protectant, moisturizer, free radical protectant, luminosity enhancer, UV protectant, collagen stimulator, depigmenting, fragrance, preservative, or denaturant, is provided

TABLE 15
Cosmetic Composition 15

| | Concentration | |
|---|---|---|
| | (% by weight with | |
| | respect to the total | |
| | weight of the | |
| Compound | composition) | |
| Ethyl Alcohol | 40-70 | solvent |
| Octocrylene | 9.5 | UV filter |
| Octisalate or | 4.9 | UV filter |
| Ethylhexyl Salicylate | | |
| Avobenzone or Butyl | 2.9 | UV filter |
| Methoxydibenzoylmethane | | |
| Diisooctyl Succinate | 5-15.0 | Emollient |
| Butyloctyl Salicylate | 1.9-5.0 | Skin conditioner |
| Water | 1.9-5.0 | Solvent |
| Ethylhexyl | 0.75-2.5 | Skin conditioner |
| Methoxycrylene | | |
| VA/Butyl | 0.75-2.5 | Film former |
| Maleate/Isobornyl | | |
| Acrylate Copolymer | | |
| Acetyl Zingerone | 0.1-0.9 | Antioxidant, |
| | | Chelating Agent |
| Ethyl Ferulate | 0.1-0.9 | Anti-oxidant |
| Polyester-8 | 0.1-0.9 | Film former |
| Caprylic/Capric | 0.1-0.9 | Skin conditioner |
| Triglyceride | | |
| Mauritia Flexuosa | 0.1-0.9 | Skin conditioner |
| (Buriti) Fruit Oil | | |
| Limnanthes Alba | 0.1-0.9 | Skin conditioner |
| (Meadowfoam) Seed Oil | | |
| Alcohol Denat. | 0.1-0.9 | Solvent |
| Glycerin | 0.1-0.9 | Humectant, Texture |
| | | Enhancer |
| Brassica Campestris | 0.1-0.9 | Skin protectant |
| (Rapeseed) Seed Oil | | |
| Rubus idaeus (Red | ≤0.1% | Skin protectant |
| Raspberry) Seed Oil | | |
| Opuntia Ficus-Indica | ≤0.1% | Skin protectant |
| Flower Extract | | |
| Methyl Propanediol | ≤0.1% | Solvent, Humectant |
| Caprylyl Glycol | ≤0.1% | Humectant |
| Phosphatidylcholine | ≤0.1% | Emollient, |
| | | Emulsifier, |
| | | Cleansing Agent |
| Vanilla Planifolia | ≤0.1% | Antioxidant, |
| Fruit Extract | | Fragrance |
| Mannitol | ≤0.1% | Humectant |
| Camellia Sinensis Leaf | ≤0.1% | Antioxidant |
| Extract | | |
| Ectoin | ≤0.1% | Anti-inflammatory, |
| | | UV protectant, |
| | | moisturizer, free |
| | | radical protectant |
| Stearic acid | ≤0.1% | Moisturizing agent |
| Palmitic Acid | ≤0.1% | Moisturizer |
| Haematococcus Pluvialis | ≤0.1% | Antioxidant, anti- |
| Extract | | inflammatory, |
| | | moisturizer, |
| | | luminosity enhancer, |
| | | UV protectant |
| Tetrahexyldecyl | ≤0.1% | Antioxidant, anti- |
| Ascorbate | | inflammatory, UV |
| | | protectant, collagen |
| | | stimulator, |
| | | depigmenting |
| Panthenol | ≤0.1% | Humectant |
| Phenylpropanol | ≤0.1% | Preservative, |
| | | Solvent |
| Bisabolo | ≤0.1% | Skin conditioner |
| Glycyrrhiza Glabra | ≤0.1% | Skin conditioner |
| (Licorice) Root Extract | | |
| Polyglyceryl-3 | ≤0.1% | Emulsifier |
| Diisostearate | | |
| Althaea Officinalis | ≤0.1% | Skin conditioner |
| Root Extract | | |
| Oryza Sativa (Rice) | ≤0.1% | Skin conditioner |
| Bran Extract | | |
| Tertiary butyl alcohol | ≤0.1% | Denaturant |
| Denatonium benzoate | ≤0.1% | Denaturant |
| Sodium benzoate | ≤0.1% | Preservative |
| Potassium benzoate | ≤0.1% | Preservative |
| Liposome | As needed | Carrier, penetration |

### Experimental Data

Photo-protective Efficacy Study: Several studies were undertaken to assess the effects of the composition in accordance with the present invention with regards to parameters associated with skin photo-aging.
Methodology: Four experimental groups were provided:
Group I: Healthy: non-irradiated skin/Untreated hOSEC
Group II: Control: photoaged skin; The distressed (photo-aged) hOSEC were obtained by daily irradiation of healthy skin with sun-like light (5 J/cm²).
Group III: Encapsulated antioxidant complex: photo aged skin+the composition in accordance with the present invention (ratio of components of the complex is: Ectoin 1% (w/v), Vitasynol 0.1% (w/v) and Seaberry blue at 2% (w/v)); Photo-aged hOSEC treated with compound under study at 2 mg/cm2.
Group IV: Free antioxidant complex: photoaged skin+free ingredients; Photo-aged hOSECs treated with compound under study at 2 mg/cm2.

Skin explant cultures (hOSEC) were used. Distress mimicking skin photo-aging was induced by daily irradiation of skin with sun-like light. The photo-protective efficacy of the test items applied topically on hOSEC was determined by measuring pro-inflammatory cytokines (IL-6 and IL-8) and matrix metalloproteinases (MMP-9) secretion. Human organotypic skin explant cultures (hOSECs) were obtained with informed consent from healthy donors undergoing plastic surgery (Authorization granted by French government ethical committee according to French law L.1245 CSP). Up to 2 hours from the surgery the skin was cut into 0.8 cm2 pieces and shipped in transport medium. Upon receipt, samples were placed with dermis facing down and epidermis facing up in culture plates containing skin culture medium without animal components supplemented with antibiotics (1% pen-strep). Tissue cultures were incubated for at least 48 hours at 37° C. under 5% CO2 for recovery prior to study initiation. In order to mimic skin photo-aging, sun-like light irradiation (5 J/cm²) was applied daily to the hOSEC. At the same time, the test products were administered topically at 2 mg/cm2, for a total of 7 applications. The test products were in contact with the hOSEC throughout the study.

Resazurin Assay: The resazurin dye (7-hydroxy-3H-phenoxazin-3-one 10-oxide) has been broadly used as an indicator of cell viability in proliferation and cytotoxicity assays. The assay is based on the ability of viable, metabolically active cells to reduce resazurin to resorufin and dihydroresorufin. This conversion is intracellular, facilitated by mitochondrial, microsomal and cytosolic oxidoreductases. Resazurin (RES) is non-toxic to cells, and it is stable in culture medium. Therefore, it allows continuous measurement of cell proliferation in vitro as either a kinetic or an endpoint assay.

Toxic insult that impairs cell viability and proliferation also affects the ability of cultures to reduce resazurin, and the rate of dye reduction is directly proportional to the number of viable cells. Therefore, as the resazurin reduction is a direct measure of the metabolic competence of cell cultures, it provides a convenient index of cell viability following product incubation both in healthy and distressed hOSEC.

Just before topical application of compounds under study, the skin explants were treated with 6 μM of resazurin solution for 1 hour. Subsequently, a volume of 100 μL sample was removed from each sample and transferred into a 96-well microplate. The resorufin formed was quantified in a fluorometer plate reader. The fluorescent signal was monitored using 530 nm excitation wavelength and 590 nm emission wavelength.

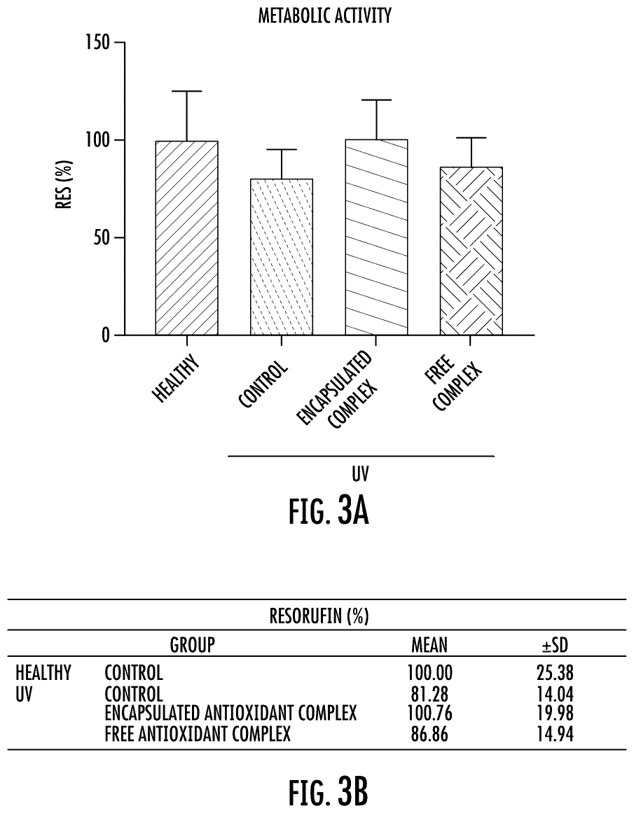

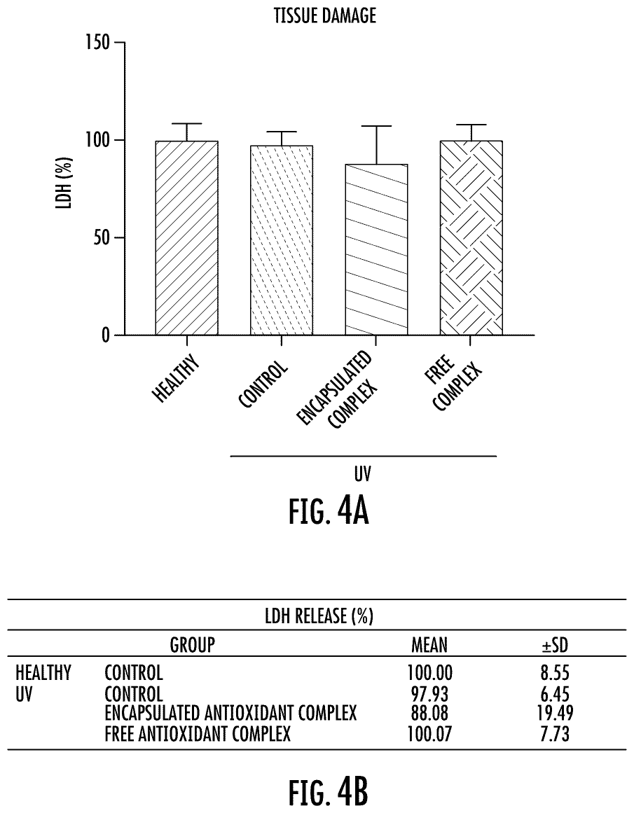

Referring to FIGS. 3A, 3B, 4A and 4B, none of the test items were found to be toxic for the human skin explants. Decrease of resazurin reduction (FIG. 3, florescent assay detecting cellular metabolic activity), nor an increase in LDH release (FIG. 4, tissue damage detection) in human skin explants compared to UV Control was observed.

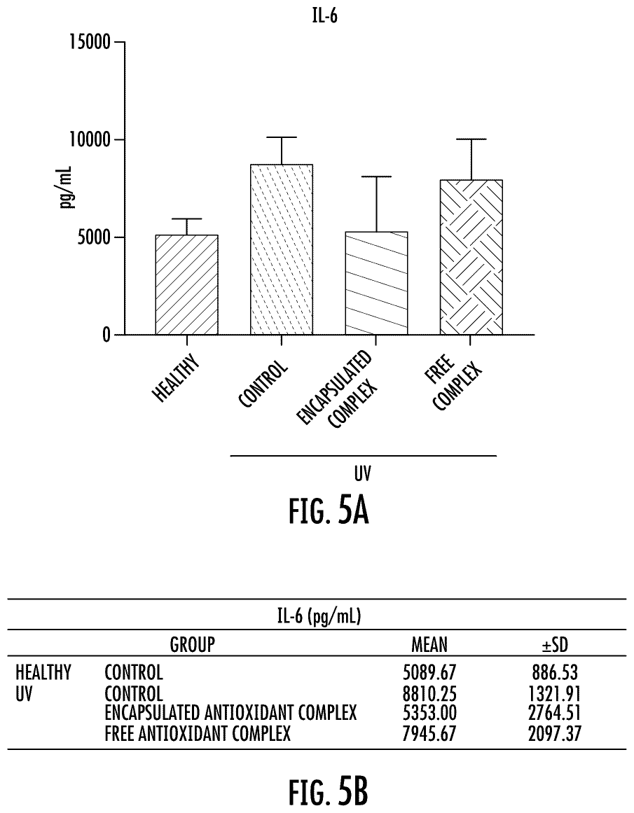

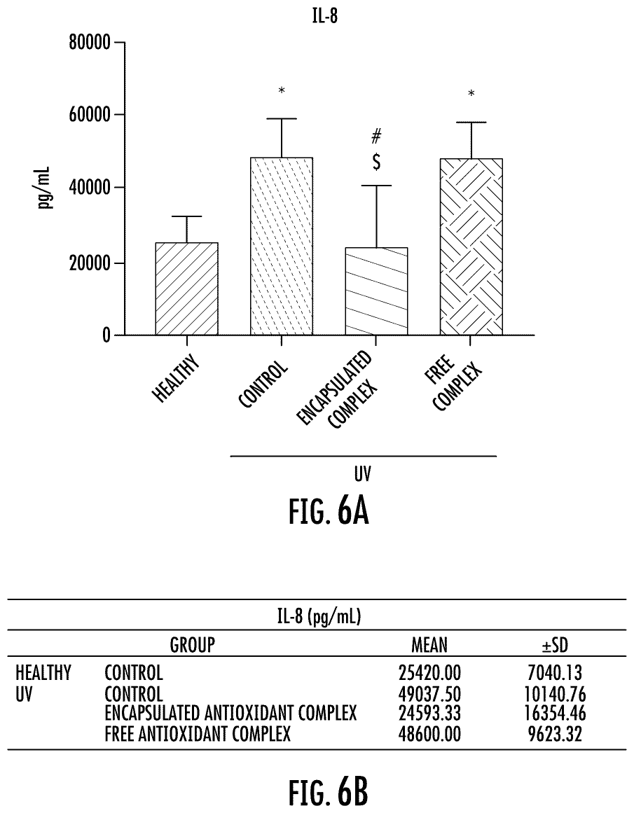

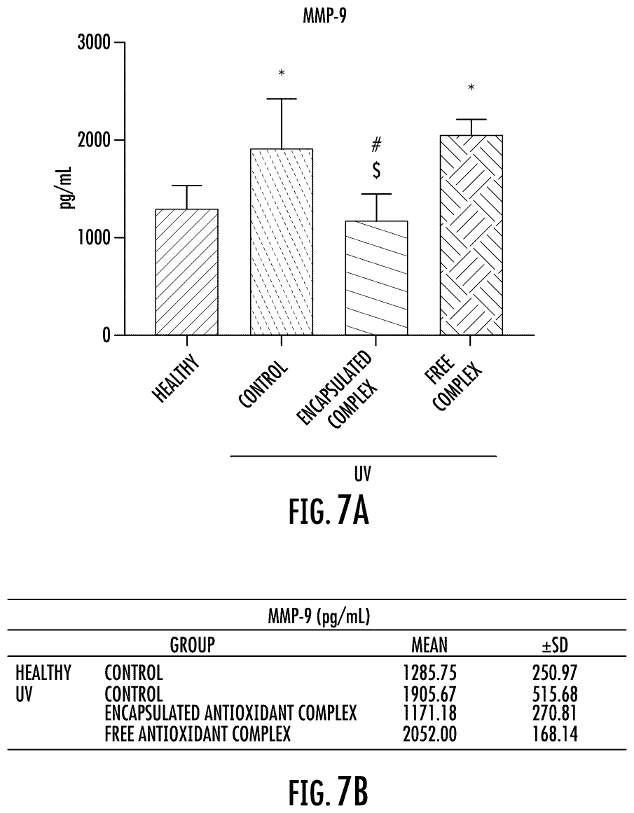

Referring to FIGS. 5A, 5B, 6A and 6B, pro-inflammatory cytokine secretion was measured. With regards to II-6, Group III, encapsulated antioxidant complex (photo aged skin+the composition in accordance with the present invention) was shown to reduce IL-6 levels by 92.92% when compared to the control group, see FIGS. 5A/5B. With regards to II-8, Group III, encapsulated antioxidant complex (photo aged skin+the composition in accordance with the present invention) was shown to reduce IL-8 levels by 103.5% when compared to the control group, see FIGS. 6A/6B. When testing for MMP-9 secretion, Group III, encapsulated antioxidant complex (photo aged skin+the composition in accordance with the present invention) was shown to reduce MMP-9 levels by 118.48% when compared to the control group, see FIGS. 7A/7B.

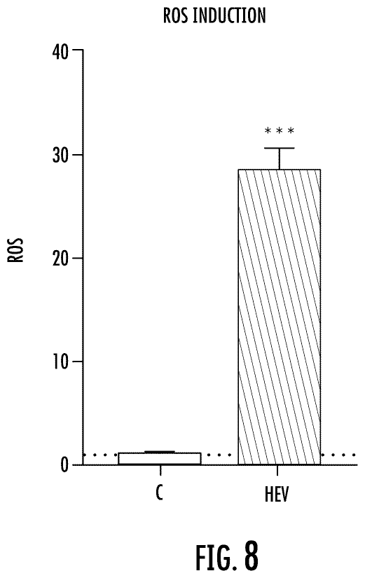

The results provided in FIGS. 5A-7B indicate that compositions in accordance with the present invention exert enhanced tissue preservation properties, and photo-protective activity in human skin under the assay conditions, compared to healthy control (untreated hOSEC) and free antioxidant complex.
HEV-induced ROS: Referring to FIGS. 8-13, ex-vivo testing was provided to assess the protective effects from High-Energy Visible (HEV) light induced reactive oxygen species (ROS) in reconstructed Human epidermis (RHE).

RHE (Reconstructed Human Epidermis 3D skin model) was acclimatized during 24 hours after reception. Tested samples were topically applied onto the surface of RHE skin tissues for 24 hours. After the incubation period, ROS reaction mix was added to RHE skin tissues. Tissues were then irradiated with Blue light or High-Energy Visible (HEV) light (Blue light or high-energy visible light (HEV) referring to wavelengths between 390 and 500 nm) during 60 minutes to induce oxidative stress and reactive oxygen species (ROS) accumulation. Non-irradiated tissue controls were kept in the dark during the irradiation period. Two hours after the irradiation process started, tissues were placed in a new plate for ROS measurement. Data were statistically analyzed.

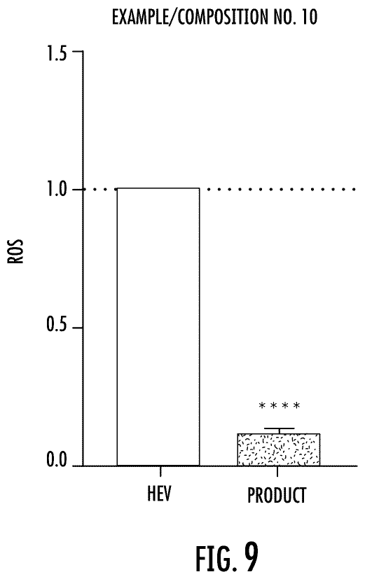

In general, HEV radiation increased ROS levels on RHE (control) by 2743±211.3%, see FIG. 8, validating the experimental system as a model of HEV-induced oxidative stress. Referring to FIG. 9, results of the HEV-induced ROS when using the composition of Example/Composition 10 are provided. Treatment with the Example/Composition 10 illustrates a 89.1±3.9% reduction/protection from HEV-induced ROS.

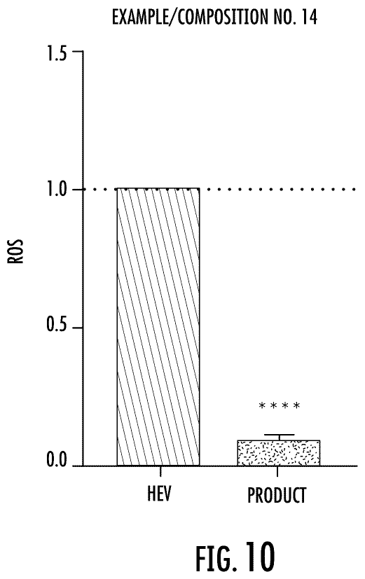

Referring to FIG. 10, results of the HEV-induced ROS when using the composition of Example/Composition 14 are provided. Treatment with the Example/Composition 14 illustrate a 90.7±3.4% reduction/protection from HEV-induced ROS.

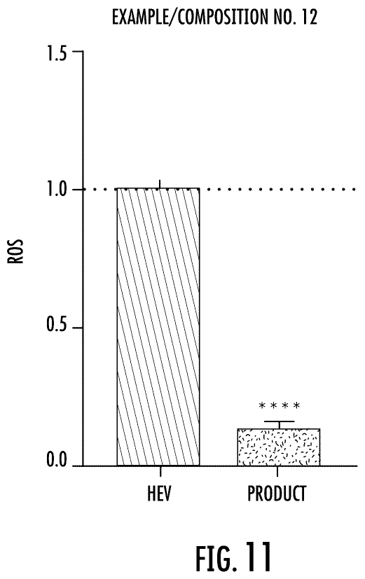

Referring to FIG. 11, results of the HEV-induced ROS when using Example/Composition 12 are provided. Treatment Example/Composition 12 illustrate a 87.0±3.9% reduction/protection from HEV-induced ROS.

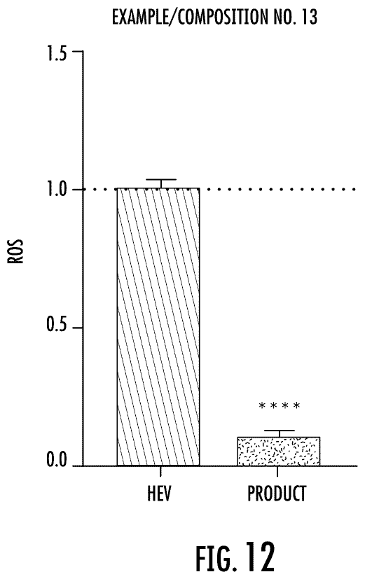

Referring to FIG. 12, results of the HEV-induced ROS when using Example/Composition 13 are provided. Treatment with Example/Composition 13 illustrate a 89.3±3.4% reduction/protection from HEV-induced ROS.

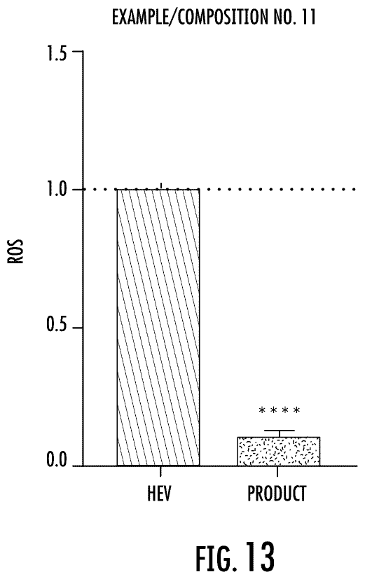

Referring to FIG. 13, results of the HEV-induced ROS when using Example/Composition 11 are provided. Treatment with the Example/Composition 11 illustrate an 89.7±4.2% reduction/protection from HEV-induced ROS.

Accordingly, all RHE treated with various compositions in accordance with the present invention exhibit protection from Reactive Oxygen Species (ROS) induced by HEV radiation (HEV-induced oxidative stress). These studies show that treatment with the products in accordance with embodiments of the invention protects from Reactive Oxygen Species (ROS) induced by HEV radiation in Reconstructed Human Epidermis (RHE).

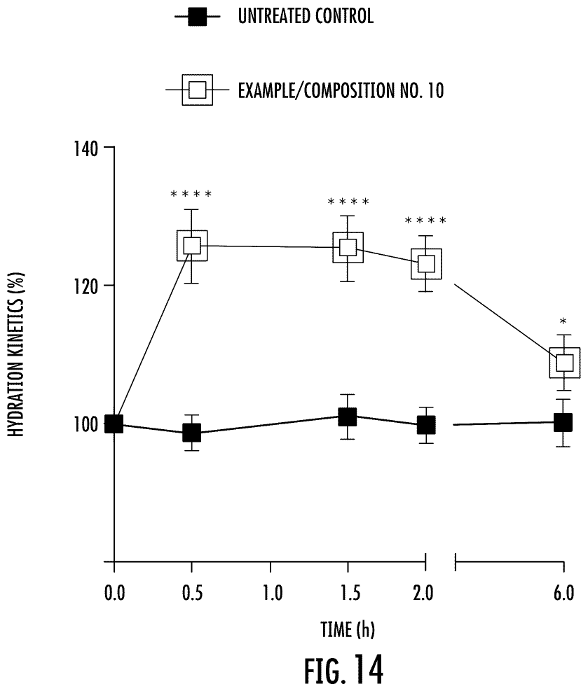

In-vivo testing: Referring to FIGS. 14-25, results for in-vivo testing on various illustrative examples in accordance with the invention are provided.

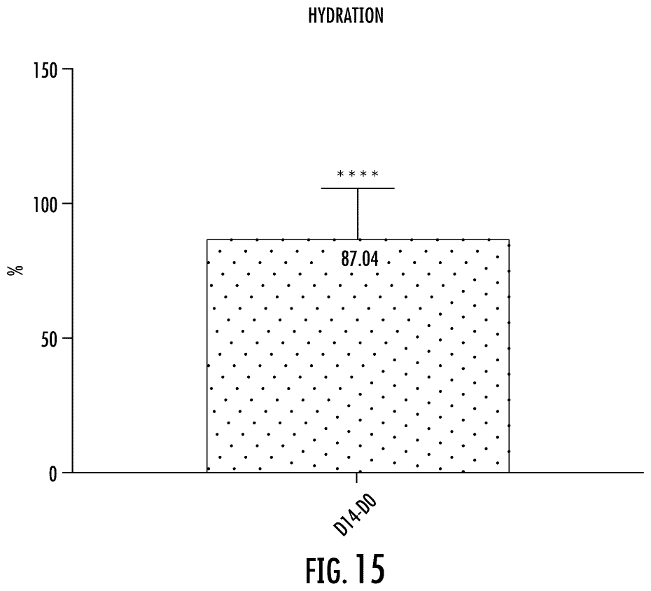

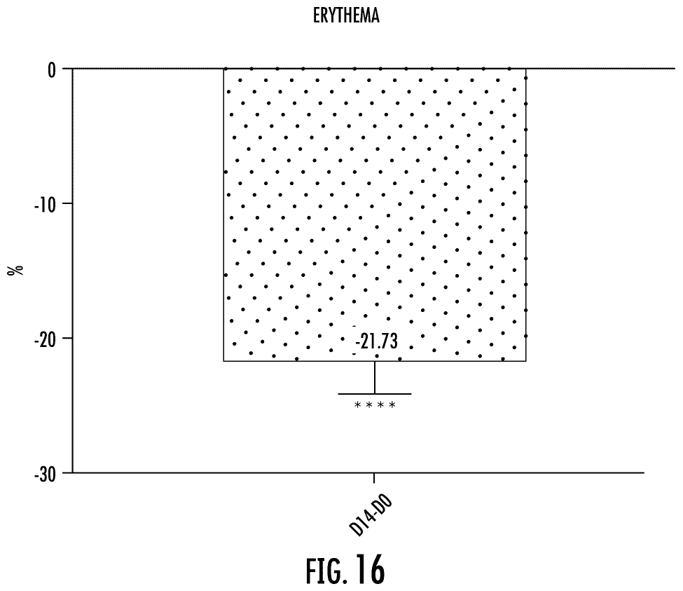

Referring to FIGS. 14-16, in-vivo testing results for Example/Composition 10, is shown. FIG. 14 illustrates increased hydration levels, vs untreated control by: 26.9% after 30 minutes; 24.4% after 1 hour, 30 minutes; 23.4% after 2 hours; and 8.6% after 6 hours. FIG. 15 illustrates the results for increased hydration levels after 14 days, increased by 87%. FIG. 16 illustrates the results for skin erythema levels after 14 days, decreased by 21.7%.

Based on a 30-person subject self-assessment test/consumer perception evaluation for Example/Composition 10, it was found that:

After the first application:
A) 93% reported to be satisfied with the treatment received
B)₉₀% agreed that product spread evenly over the skin
C) 90% agreed that product did not leave a white cast
D) 90% agreed that product hydrates the skin quickly
E) 83% reported that product has a light texture that feels comfortable on the skin
F) 80% agreed that skin appears healthier after using this product.

After 6 hours of application
A) 93% reported that product helps support the skin's moisture barrier
B) 90% agreed that product helps skin feel soft and hydrated all day
C) 80% reported that product is compatible with sensitive or problematic skin
D) 87% agreed that skin appears healthier after using this product
E) 80% agreed that product felt lightweight all day
F) 73% agreed that product helps neutralize redness.

After 14 days of Application
A) 97% agreed that skin feels moisturized after application
B) 93% agreed that product helps skin feel soft and hydrated all day
C) 93% reported that product helps support the skin's moisture barrier
D) 83% reported product is compatible with sensitive or problematic skin
E) 83% agreed that product helps neutralize redness
F) 80% agreed that product is not heavy or sticky.

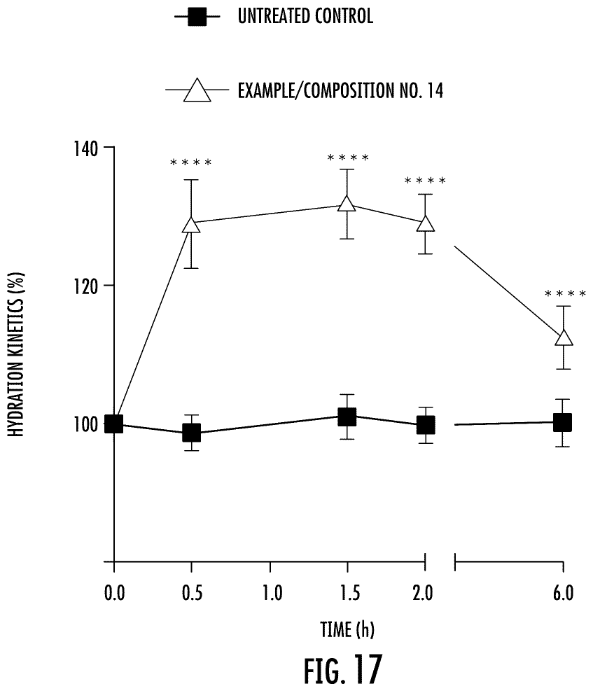

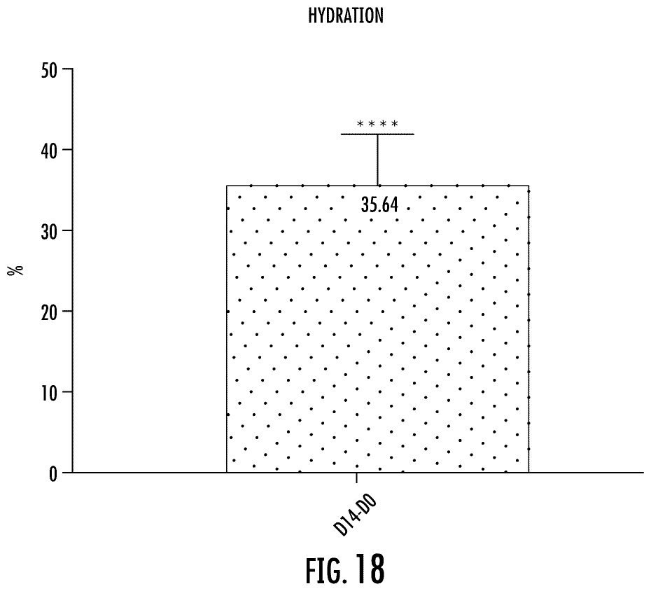

Referring to FIGS. 17-18, in-vivo testing results for Example/Composition 14 is shown. FIG. 17 illustrates increased hydration levels, vs untreated control by: 30.0% after 30 minutes; 30.5% after 1 hour, 30 minutes; 29.0% after 2 hours; and 12.3% after 6 hours. FIG. 18 illustrates the results for increased hydration levels after 14 days, increased by 35.6%.

Based on a 30-person subject self-assessment test/consumer perception evaluation for Example/Composition 14, it was found that:
After the first application:
A) 93% agreed that skin felt moisturized after application
B) 90% agreed that product spread evenly over the skin
C) 90% agreed that product hydrates the skin
D) 87% agreed that product feels nourishing on the skin
E)₈₃% agreed that product blends seamlessly into the skin
F) 83% agreed that product did not leave a white cast.

After 6 hours of application
A) 97% reported that skin feels protected from sun exposure
B) 87% agreed that product hydrates the skin
C) 83% agreed that product feels nourishing on the skin
D) 83% agreed that skin felt moisturized after application
E) 80% agreed that product blends seamlessly into the skin
F) 77% agreed that product would be used daily.

After 14 days of Application
A) 90% reported that skin feels protected from sun exposure
B) 87% agreed that product feels nourishing on the skin
C) 87% agreed that product hydrates the skin
D) 80% agreed that product spread evenly over the skin
E) 80% agreed that product blends seamlessly on the skin
F) 80% agreed that product leaves skin soft and smooth.

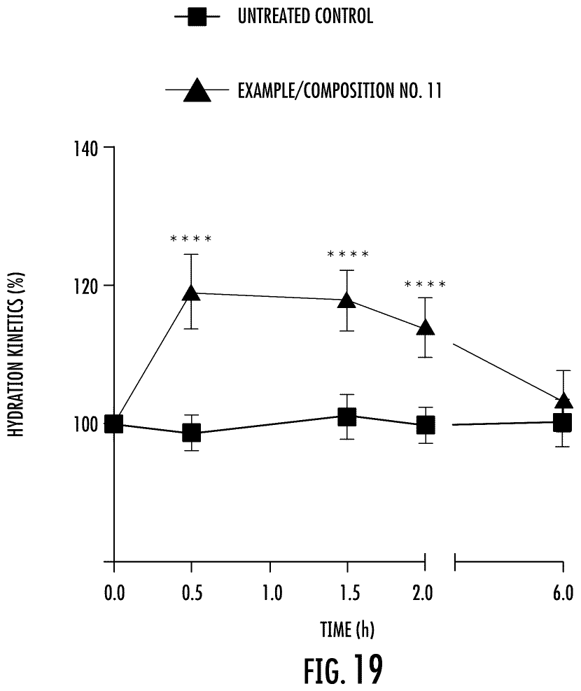

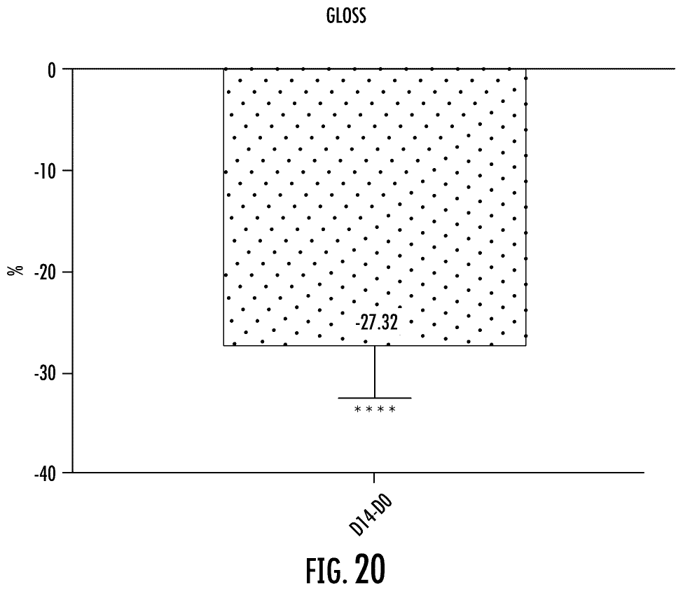

Referring to FIGS. 19-20, in-vivo testing results for Example/Composition 11 is shown. FIG. 19 illustrates an increased hydration levels, vs untreated control by: 20.2% after 30 minutes; 16.7% after 1 hour, 30 minutes; 14% after 2 hours; and hydration levels are maintained at 6 hours. FIG. 20 illustrates the results for gloss levels (mattifying effect) after 14 days, decrease 27.3%.

Based on a 30-person subject self-assessment test/consumer perception evaluation for Example/Composition 11, it was found that:
After the first application:
A) 100% reported to be satisfied with the treatment received
B)₉₇% agreed that product spread evenly over the skin
C) 93% agreed that product applies sheer
D) 90% agreed that product did not leave a white cast
E) 80% agreed they would use this product daily
F) 87% agreed that the product did not leave skin feeling tight or dry.

After 6 hours of application
A) 93% agreed that product felt light and smooth on the skin
B) 90% agreed that the product did not leave skin feeling tight or dry
C) 87% agreed that product did not leave a white cast
D) 83% agreed that product feels lightweight
E) 80% agreed that they would use this product daily
F) 80% reported that skin appears mattified and smooth.

After 14 hours of application
A) 90% agreed that product provides a soft-matte finish
B) 87% reported that skin appears mattified and smooth
C) 80% agreed that product minimizes the appearance of shine
D) 80% agreed that product felt light and smooth on the skin
E) 80% agreed that the product leaves skin with a natural-looking matte finish
F) 80% agreed that keeps skin feeling mattified throughout the day.

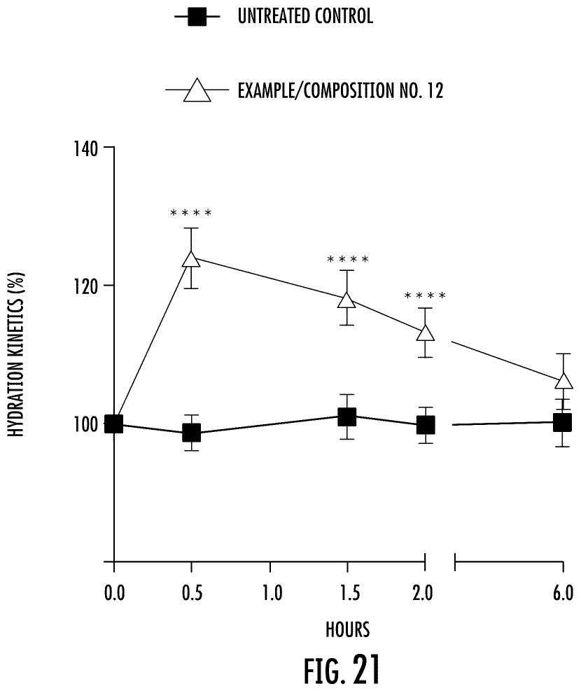

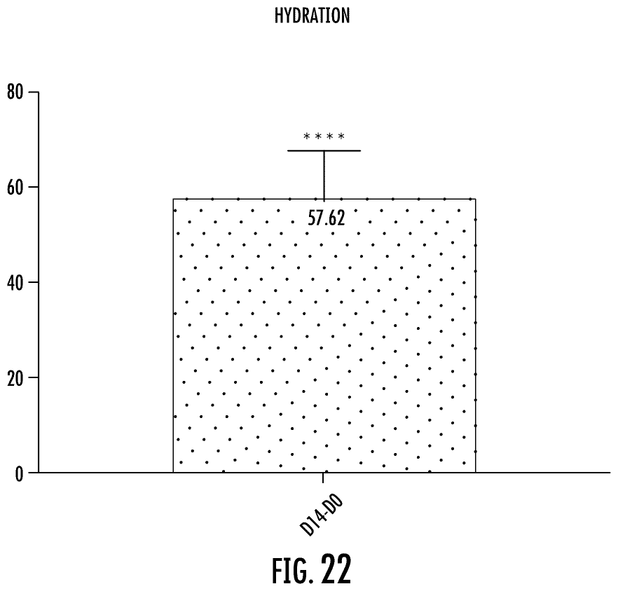

Referring to FIGS. 21-22, in-vivo testing results for Example/Composition 12 is shown. FIG. 21 illustrates increased hydration levels, vs untreated control by: 25.0% after 30 minutes; 17.0% after 1 hour, 30 minutes; 13.3% after 2 hours; and hydration levels are maintained at 6 hours. FIG. 22 illustrates the results for hydration levels after 14 days, increased by 57.6%.

Based on a 30-person subject self-assessment test/consumer perception evaluation for Example/Composition 12, it was found that:
After the first application:
A) 83% agreed that product felt weightless
B) 83% agreed that product applies easily
C) 80% agreed that product leaves skin soft and smooth
D) 77% agreed that product is not heavy and sticky
E) 73% agreed that product blends invisibly on the skin.

After 6 hours of application
A) 80% reported that product felt light and smooth on the skin
B) 83% agreed that product felt weightless
C) 83% agreed that product applies easily
D) 80% agreed that product hydrates the skin
E) 80% agreed that skin felt moisturized after application.

After 14 days of application
A) 97% reported to be satisfied with the treatment received
B) 90% reported that skin feels protected from sun exposure
C) 80% agreed that product felt weightless
D) 70% agreed that product leaves skin soft and smooth.

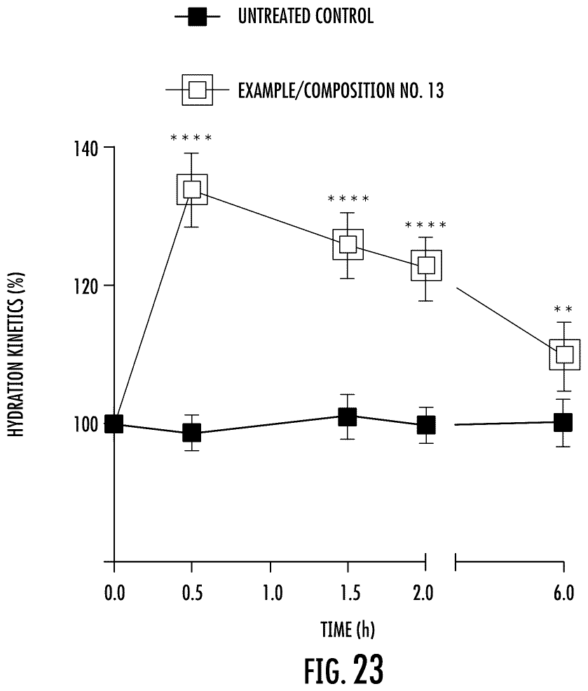

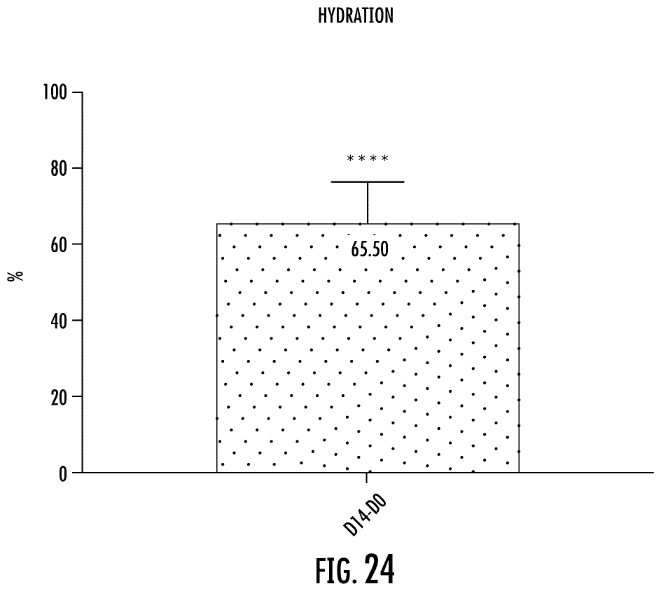

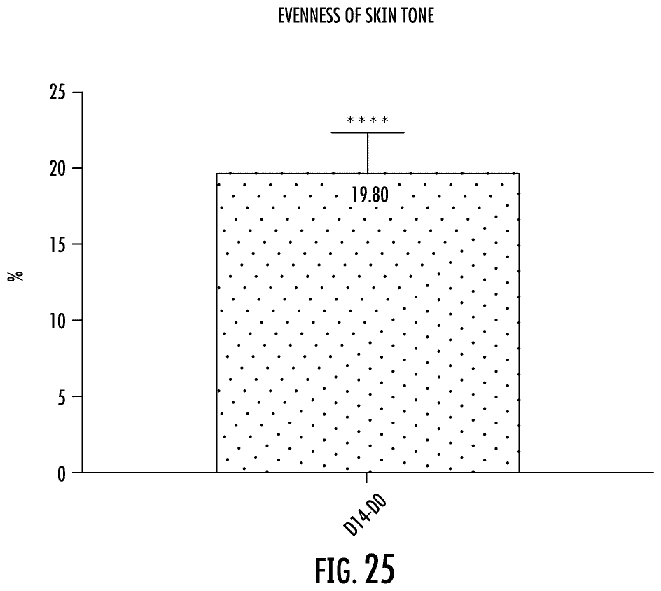

Referring to FIGS. 23-25, in-vivo testing results for Example/Composition 13 is shown. FIG. 23 illustrates increased hydration levels, vs untreated control by: 34.8% after 30 minutes; 24.6% after 1 hour, 30 minutes; 22.5% after 2 hours; and 9.3% after 6 hours. FIG. 24 illustrates the results for hydration levels after 14 days increased by 65.5%. FIG. 25 illustrates testing relating to evenness of skin tone, increased by 19.8% after 14 days.

Based on a 30-person subject self-assessment test/consumer perception evaluation for Example/Composition 13, it was found that:
After first application
A) 93% agreed that product blends seamlessly on the skin
B) 93% agreed that product leaves a natural finish on the skin
C) 90% agreed that product makes the skin look more even toned without makeup
D) 80% agreed that product gives the skin a radiant finish
E) 87% agreed that product helps even skin tone
F) 80% agreed that product gives skin a healthy glow.

After 6 hours of application
A) 93% agreed that product blends seamlessly on the skin
B) 90% agreed that product felt light and smooth on the skin
C) 90% agreed that product helps even skin tone
D) 87% agreed that product gives skin a healthy glow
E) 87% agreed that product smooths skin texture
F) 80% agreed that product hydrates the skin
G) 77% agreed that product gives the skin a radiant finish.

After 14 days of application
A) 93% agreed that product blends seamlessly on the skin
B) 93% agreed that product gives the skin a radiant finish
C) 93% agreed that product makes the skin look more even toned without makeup
D) 93% agreed that product helps even skin tone
E) 87% agreed that product gives skin a healthy glow
F) 80% agreed that skin appears healthier after using this product.

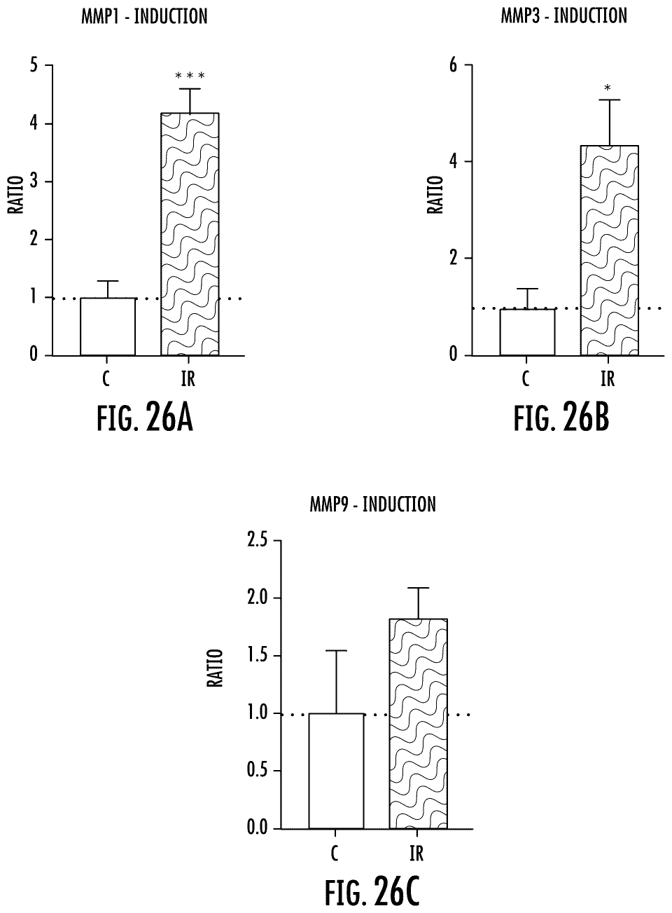

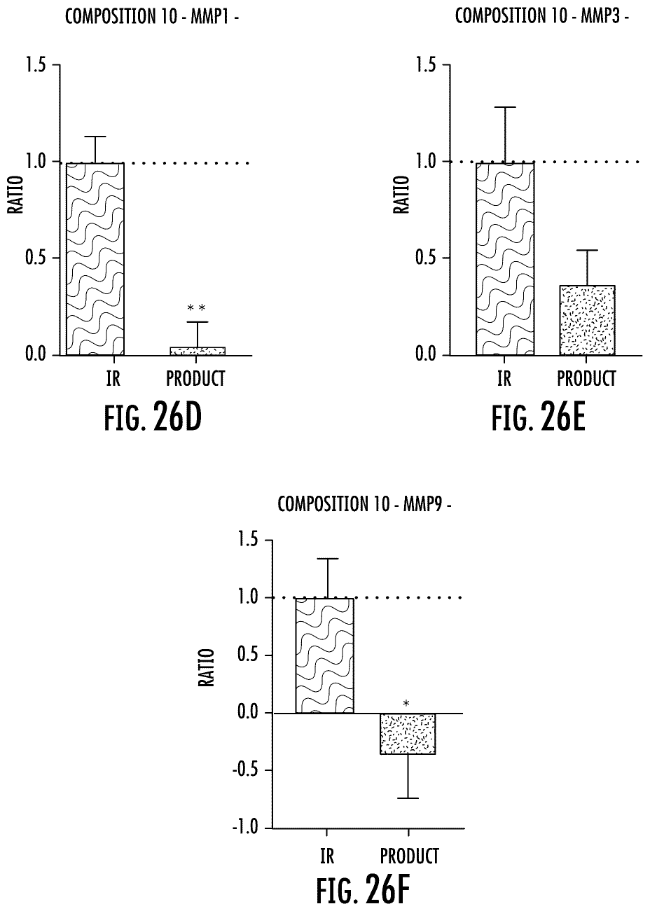

Referring to FIGS. 26A-26F, results in the analysis of the regulation of matrix metalloproteinases after infrared radiation (IR) in human epidermis of Example/Composition 10 are shown. The study was undertaken to evaluate the regulation of matrix metalloproteinases (MMP1, MMP3 and MMP9) by qRT-PCR analysis, after infrared radiation in Reconstructed Human Epidermis (RHE). EPIDERM™ Reconstructed Human Epidermis (size 0.33 cm2 in 24-well plates) was acclimatized during 24 hours (hrs) after reception, following manufacturer's instructions. Tested sample was topically applied onto the surface of RHE for 24 hrs. After the treatment, tissues were irradiated with IR radiation for 30 min. After 3 hours of incubation after the irradiation, total RNA was extracted using RNeasy kit (Qiagen) and treated with DNAse-I to remove any contamination from genomic DNA. RNA quality and quantity were checked in a Nano-Drop spectrophotometer, and 500 ng of total RNA was used to synthesize cDNA, using First-strand Synthesis kit (TaKaRa). Finally, quantitative PCR (qPCR) was performed in a real-time PCR machine (QuantStudio 5, Applied BioSystem). To perform raw data analysis, the 2-ΔΔCt method (Livak & Schmittgen, 2001) was used to calculate the gene relative expression ratio to nontreated control (C). Actin (ACT) was used as a reference housekeeping gene.

The results of the study showed that IR radiation significantly increased gene expression levels of MMP1 (FIG. 26A) 316.6±53.0%, and MMP3 (FIG. 26B) 336.2±1-2.7%. The results for MMP9 (FIG. 26C) were not statistically significant, but showed an increase as well. Example/Composition 10 reduced the IR-induced MMP1 gene expression levels by 96.6±19.6% (FIG. 26D) and MMP 9 gene expression by 134.8±53.3% (FIG. 26E). The results for MMP3 were not significant, but showed a decrease as well, (64.6, see FIG. 26F). The study indicates that treatment with Example/Composition 10 regulates MMP1, MMP3, and MMP 9, after radiation, in RHE.

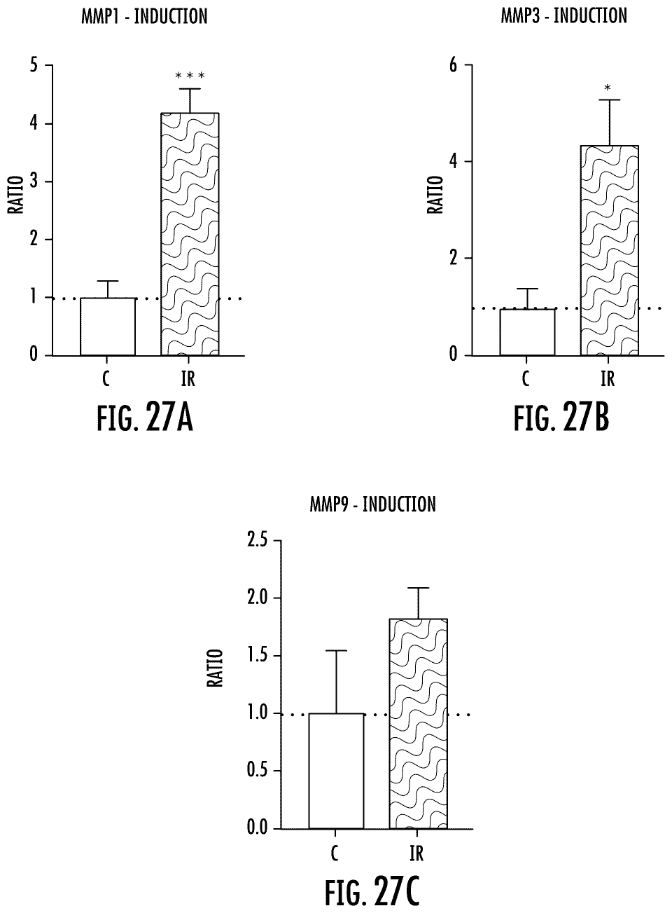

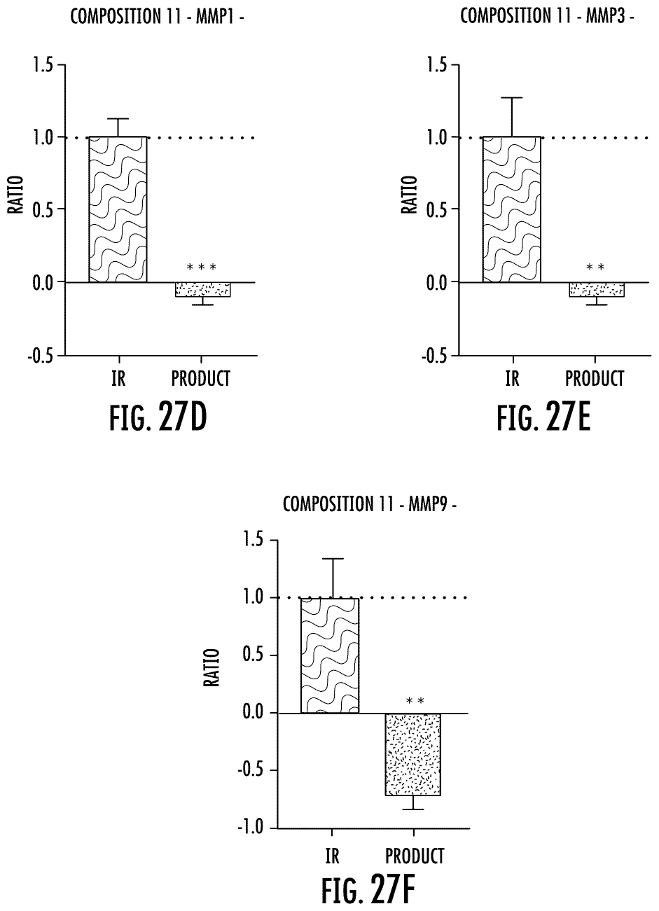

Referring to FIGS. 27A-27F, results in the analysis of the regulation of matrix metalloproteinases after infrared radiation (IR) in human epidermis of Example/Composition 11 are shown. The study was undertaken to evaluate the regulation of matrix metalloproteinases (MMP1, MMP3 and MMP9) by qRT-PCR analysis, after infrared radiation in Reconstructed Human Epidermis (RHE). EPIDERM™ Reconstructed Human Epidermis (size 0.33 cm2 in 24-well plates) was acclimatized during 24 hours (hrs) after reception, following manufacturer's instructions. Tested sample was topically applied onto the surface of RHE for 24 hrs. After the treatment, tissues were irradiated with IR radiation for 30 min. After 3 hours of incubation after the irradiation, total RNA was extracted using RNeasy kit (Qiagen) and treated with DNAse-I to remove any contamination from genomic DNA. RNA quality and quantity were checked in a Nano-Drop spectrophotometer, and 500 ng of total RNA was used to synthesize cDNA, using First-strand Synthesis kit (TaKaRa). Finally, quantitative PCR (qPCR) was performed in a real-time PCR machine (QuantStudio 5, Applied BioSystem). To perform raw data analysis, the 2-ΔΔCt method (Livak & Schmittgen, 2001) was used to calculate the gene relative expression ratio to nontreated control (C). Actin (ACT) was used as a reference housekeeping gene.

The results of this study showed that IR radiation significantly increased gene expression levels in MMP1 (FIG. 27A) by 316.6±53.0%, and MMP3 (FIG. 27B) by 336.2±102.7%. The results for MMP9 (FIG. 27C) were not statistically significant, but increased as well. Example/Composition 11 reduced the IR-induced MMP1 gene expression levels by 110.1±14.7% (FIG. 27D), MMP3 gene expression levels by 110.3±28.8% (FIG. 27E), and MMP 9 gene expression levels by 170.4±37.3% (FIG. 26E). The study indicates that treatment with Example/Composition 11 regulates MMP1, MMP3, and MMP 9, after radiation, in RHE.

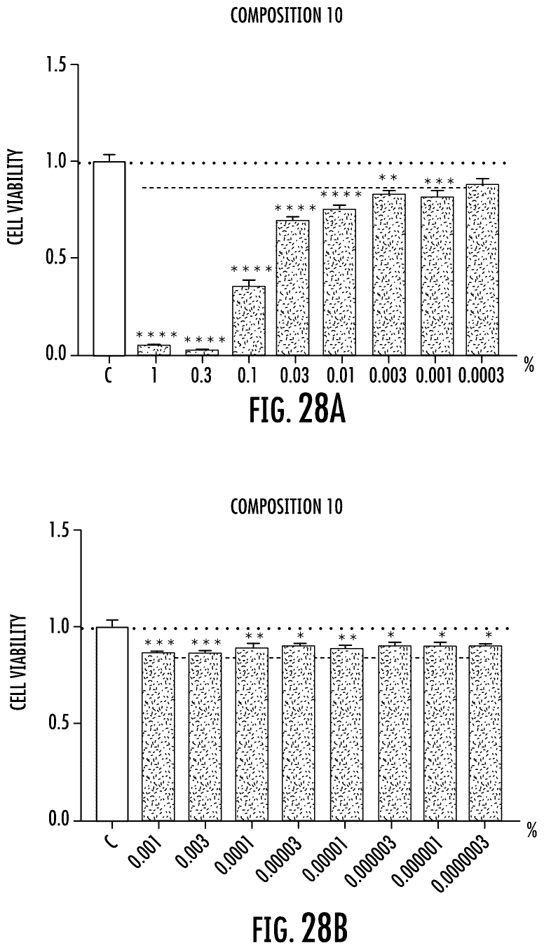

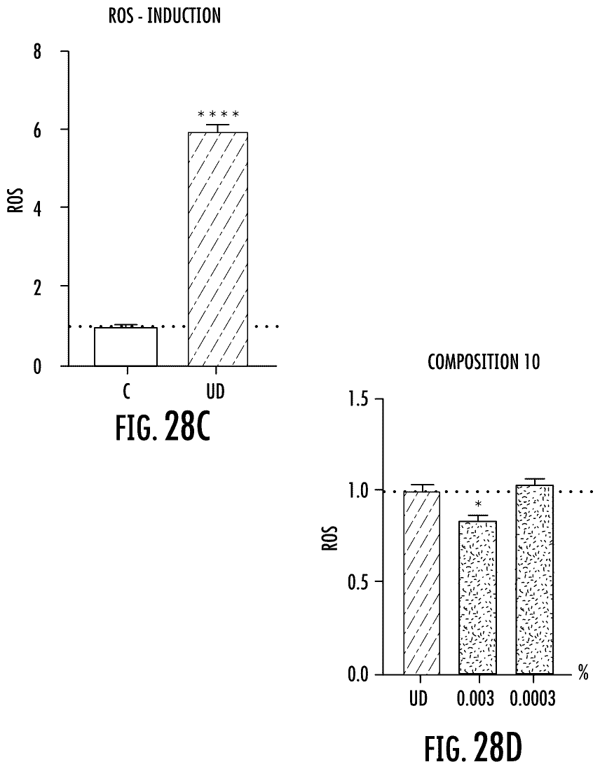

Referring to FIGS. 28A-28D, results in the analysis of the protective effects against pollution-induced reactive Oxygen Species (ROS) in human keratinocytes of Example/Composition 10 are shown. The study was undertaken to evaluate the protective effects of Example/Composition 10 against pollution induced ROS in human keratinocytes.

Cell numbers and viability were determined using Trypan-Blue staining and counting in a Bürker chamber under the microscope. For the MTT viability assay, human keratinocytes were cultured overnight at a 10.000 cells/well density in a 96 well plate, in supplemented growth medium. 24 hrs later, the culture medium was replaced with fresh medium containing the tested product at 8 different concentrations (1, 0.3, 0.1, 0.03, 0.01, 0.003, 0.001 and 0.0003%). After 24 hrs of incubation, the medium was removed, and MTT solution was added to each well. Plates were incubated at 37° C. for 3 hrs. MTT reagent was removed and DMSO at 100% was added to each well to solubilize formazan crystals, then the absorbance was measured at 550 nm and 620 nm as a reference on a scanning multi-well spectrophotometer. In a second experiment, the MTT viability assay was repeated with lower concentrations of the tested product (0.001-0.0003-0.0001-0.00003-0.00001-0.000003-0.000001-0.0000003%).

ROS quantification: Human keratinocytes were cultured overnight at a 10.000 cells/well density in a black 96-well plate, in growth media. 24 hrs later, the culture media was removed and replaced by new culture medium supplied with Urban Dust and composition 10 at 0.003% and 0.0003% concentrations. After additional 24 hrs of incubation, PBS and ROS master mix were added in all cultured wells. Two hours after ROS master mix addition to cells, ROS levels were measured in all samples. The intracellular ROS react with a fluorogenic sensor localized in the cytoplasm, resulting in a fluorescent product whose appearance is proportional to ROS levels. Fluorescence quantification was measured at □ex=490/em=525. In parallel, an MTT assay was performed under the same conditions to correct ROS levels fluctuations due to changes in cell viability.

Results (cell viability) showed that when cells were treated with Example/Composition 10, cell viability reached a plateau (see red dashed line, FIG. 28A) after a concentration of 0.01%. Following the OECD guidelines for cell culture toxicity assays in which a 25% margin of safety is used, a lower non-toxic threshold was established to determine the working concentrations for the analysis (FIG. 28B). Based on these results, the selected working concentrations for the antioxidant assay were 0.003% and 0.0003%.

FIG. 28C shows that UD exposure significantly increased ROS levels in human keratinocytes by 493.5±19.0%.

Previous treatment of the cells with the Example/Composition 10 resulted in, Example/Composition 10 at 0.003% protected from UD-induced ROS by 15.8±4.6%, see FIG. 28D.

Accordingly, the present shows that treatment with the Example/Composition 10 protects from ROS induced by UD exposure in human keratinocytes.

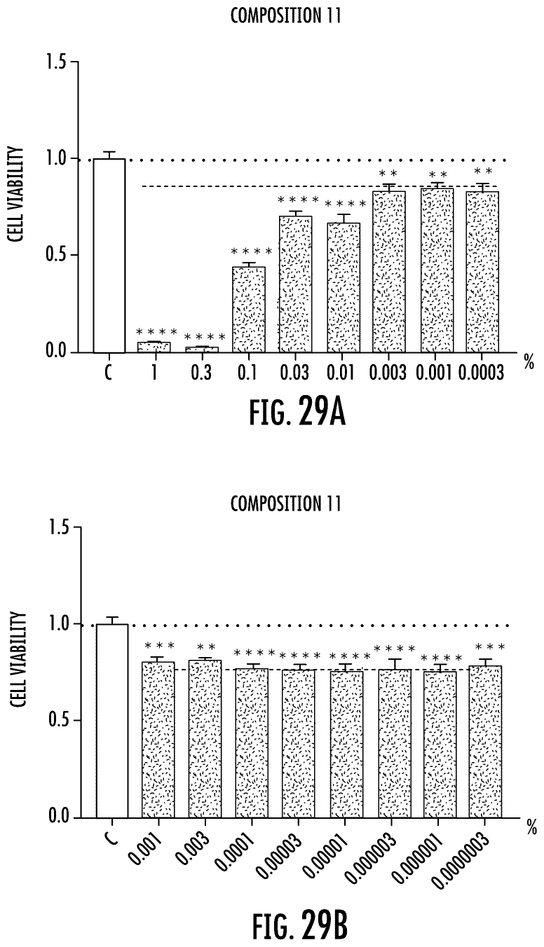

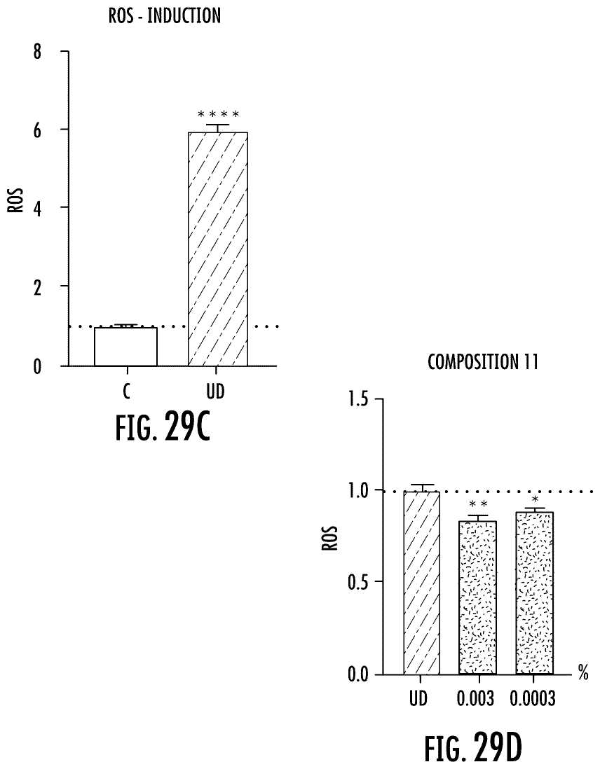

Referring to FIGS. 29A-29D, results in the analysis of the protective effects against pollution-induced reactive Oxygen Species (ROS) in human keratinocytes of Example/Composition 11 are shown. The study was undertaken to evaluate the protective effects of Example/Composition 11 against pollution induced ROS in human keratinocytes.

Cell numbers and viability were determined using Trypan-Blue staining and counting in a Burker chamber under the microscope. For the MTT viability assay, human keratinocytes were cultured overnight at a 10.000 cells/well density in a 96 well plate, in supplemented growth medium. 24 hrs later, the culture medium was replaced with fresh medium containing the tested product at 8 different concentrations (1, 0.3, 0.1, 0.03, 0.01, 0.003, 0.001 and 0.0003%). After 24 hrs of incubation, the medium was removed, and MTT solution was added to each well. Plates were incubated at 37° C. for 3 hrs. MTT reagent was removed and DMSO at 100% was added to each well to solubilize formazan crystals, then the absorbance was measured at 550 nm and 620 nm as a reference on a scanning multi-well spectrophotometer. In a second experiment, the MTT viability assay was repeated with lower concentrations of the tested product (0.001-0.0003-0.0001-0.00003-0.00001-0.000003-0.000001-0.0000003%).

ROS quantification: Human keratinocytes were cultured overnight at a 10.000 cells/well density in a black 96-well plate, in growth media. 24 hrs later, the culture media was removed and replaced by new culture medium supplied with Urban Dust and composition 10 at 0.003% and 0.0003% concentrations. After additional 24 hrs of incubation, PBS and ROS master mix were added in all cultured wells. Two hours after ROS master mix addition to cells, ROS levels were measured in all samples. The intracellular ROS react with a fluorogenic sensor localized in the cytoplasm, resulting in a fluorescent product whose appearance is proportional to ROS levels. Fluorescence quantification was measured at □ex=490/em=525. In parallel, an MTT assay was performed under the same conditions to correct ROS levels fluctuations due to changes in cell viability.

Results (cell viability) showed that when cells were treated with the Example/Composition 11, cell viability reached a plateau (see red dashed line, FIG. 29A) after a concentration of 0.01%. Following the OECD guidelines for cell culture toxicity assays in which a 25% margin of safety is used, a lower non-toxic threshold was established to determine the working concentrations for the analysis (FIG. 29B). Based on these results, the selected working concentrations for the antioxidant assay were 0.003% and 0.0003%.

FIG. 29C shows that UD exposure significantly increased ROS levels in human keratinocytes by 493.5±19.0% compared to the non-treated control.

Previous treatment of the cells with Example/Composition 11 resulted in, Example/Composition 11 at 0.003% and 0.0003% protected from UD-induced ROS by 15.3±4.0% and 11.4±4.0%, respectively, see FIG. 29D.

Accordingly, the present shows that treatment with the composition 11 protects from ROS induced by UD exposure in human keratinocytes.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A cosmetic or personal care composition comprising:
a natural antioxidant;
tetrahexyldecyl ascorbate;
ectoin; and
an encapsulating delivery vesicle which delivers one or more ingredients of said composition to a target area.

2. The composition according to claim 1, further including one or more additional skin care related functional ingredients.

3. The composition according to claim 1, wherein the natural antioxidant is Astaxanthin.

4. The composition according to claim 1, wherein said natural antioxidant is an extract of Haematococcus pluvialis.

5. The composition according to claim 4, wherein said H. pluvalis extract includes Caprylic Capric Triglyceride.

6. The composition according to claim 5, wherein a concentration of said Caprylic Capric Triglyceride is greater than 50%, % mass fraction, and a concertation of said Haematococcus Pluvialis Extract is between 5.0%-9.9%, % mass fraction.

7. The composition according to claim 2, wherein said one or more additional skin care related functional ingredients comprise, a solvent, an emollient, a texture enhancing ingredient, a UV filter, a humectant, a skin hydration enhancer, a carrier, a filler, an emulsion stabilizer, an opacifying agent, a moisturizing agent, an emulsifier, an antioxidant, a stabilizer, a conditioning agent, a water-binder, a viscosity agent, a skin conditioner, a preservative, a mineral dye, a mild cleansing agent, a skin-softening agent, an occlusive, a hydrating agent, a fragrance, a viscosity-decreasing agent, a masking agent; a whitening agent, a binding agent, a buffering agent, a pH controller, an anti-inflammatory; free radical protectant; a luminosity enhancer, a collagen stimulator, a depigmenting agent, an abrasive, an anti-caking agent, an anti-bulking agent, a pH adjuster, a color additive, a skin protector from UV damage, a UV rays absorber, conditioning agent, a short-wave UVB ray absorber, a UV Filter, a sunscreen agent that works primarily in the UVB range, a texture enhancing ingredient, a surfactant, a synthetic texture-enhancing ingredient, a suspending or dispersing agent, an emulsion stabilizer, a film-former, a texture-enhancing thickener, a viscosity increasing agent, a chelating agent, a protective and natural skin conditioning agent, a synthetic skin-softening agent, an abrasive, an absorbent, an anticaking agent, a bulking agent, a viscosity controller, an anti-inflammatory, a free radical protectant, a luminosity enhancer, a collagen stimulator, a depigmenting agent, a cell generation restorer, an elasticity improver, or combinations thereof.

8. The composition according to claim 1, wherein said encapsulating delivery vesicle is a liposome.

9. The composition according to claim 8, wherein said liposome comprises a diameter particle size of 50-500 nm.

10. The composition according to claim 8, wherein said liposome comprises omega 9 fatty acids, omega 6 fatty acids, or combinations thereof.

11. The composition according to claim 5, wherein said liposome comprises phytostherols, carotenoids, vitamins xanthophylls, or combinations thereof.

12. A cosmetic or personal care composition comprising:
a delivery vesicle which delivers one or more encapsulated ingredients to a target area, said encapsulated ingredients comprising an extract of Haematococcus pluvialis, tetrahexyldecyl ascorbate, and ectoin; and
non-encapsulated ingredients, said non-encapsulated ingredients comprising one or more skin care related functional ingredients.

13. The composition according to claim 12, wherein the concentration of said Haematococcus pluvialis extract is between 0.10% and 0.2% by weight, the concentration of Tetrahexyldecyl ascorbate is between 0.05 and 0.15% by weight, and the concentration of ectoin is between 0.50 and 1.50% by weight.

14. A method of delivering a cosmetic or personal care composition comprising:
delivering to a target area, a cosmetic or personal care composition, said composition comprising a delivery vesicle which delivers one or more encapsulated ingredients to a target area, said encapsulated ingredients comprising an extract of Haematococcus pluvialis, tetrahexyldecyl ascorbate, and ectoin; and non-encapsulated ingredients, said non-encapsulated ingredients comprising one or more skin care related functional ingredients.

15. The method according to claim 14, wherein said one or more encapsulated ingredients are stablely passed through a stratum corneum layer to exert their effects in an epidermal layer.
